(19) United States
(12) Patent Application Publication
(10) Pub. No.: US 2015/0210717 A1
(43) Pub. Date: Jul. 30, 2015

(54) SPECIFIC INHIBITORS OF PROTEIN P21 AS THERAPEUTIC AGENTS

(71) Applicant: BADEN-WÜRTTEMBERG STIFTUNG GMBH, Stuttgart (DE)

(72) Inventors: Cagatay Günes, Ulm (DE); Elena Marita Hoffman, Goppingen (DE); Karl Lenhard Rudolph, Ulm (DE)

(21) Appl. No.: 14/427,444

(22) PCT Filed: Sep. 13, 2013
(86) PCT No.: PCT/EP2013/069014

§ 371 Date: Mar. 11, 2015

(30) Foreign Application Priority Data
Sep. 13, 2012 (EP) 12 184 310.6

(51) Int. Cl. C07D 513/10; C07D 498/04; C07D 471/16; C07D 209/88; C07D 221/14; C07D 249/14; C07C 235/56; C07D 487/04; C07D 471/04

(52) U.S. Cl. CPC C07D 513/10; C07D 487/04; C07D 498/04; C07D 471/16; C07D 471/04; C07D 221/14; C07D 249/14; C07C 235/56; C07D 209/88

(57) ABSTRACT

The present invention relates to novel inhibitors of p21. These inhibitors are useful as therapeutic agents for promot ing cell regeneration and in the treatment of cancer. Improvement of cell regeneration is particularly desirable in patients of old age or in patients suffering from chronic diseases, acute or chronic injuries.

The present invention relates to novel inhibitors of p21. These inhibitors are useful as therapeutic agents for promoting cell regeneration and in the treatment of cancer. Improvement of cell regeneration is particularly desirable in patients of old age or in patients suffering from chronic diseases or acute injuries.

### BACKGROUND OF THE INVENTION

p21 (also known as cyclin dependent kinase inhibitor 1A: CDKN1A) is a negative regulator of the cell cycle and its expression is increased when damages to DNA or telomere dysfunction or other types of cellular stresses occur, such as oxidative stress or replication stress. In these cases, the expression of p21 leads to a temporary cell cycle arrest (in the case of reparable DNA damages) or to a permanent cell cycle arrest (in the case of telomere dysfunction). Cell cycle arrest in response to DNA damage or other stresses is dependent on p21, and the genetic deletion of p21 enables cells to continue with cell division for a few cycles although the telomeres are damaged (Brown J. P. et al. (1997) Science, 277(5327): 831-834). In a mouse model, this effect led to an improvement of tissue preservation in aging mice with dysfunctional telomeres (Choudhury A. R. et al. (2007) Nat. Genet. 39(1): 99-105).

Dysfunction of telomeres and an activation of p21 also occur in the context of aging and suppression of regeneration caused by chronic diseases in humans (Kuwano K. et al. (1996) Am. J. Respir. Crit. Care Med., 154 (2 Pt 1): 477-483; Lunz J. G. 3rd et al. (2005) Hepatology 41(6): 1262-1271; Djojosubroto M. W. et al. (2005) Hepatology 42(5): 1127-1136). On the basis of this scientific background information, the inhibition of p21 might be a promising therapeutic approach for the improvement of regeneration in the context of aging and chronic diseases. Several fundamental aspects have to be considered to achieve this goal, though. The expression of p21 is activated by the tumor suppressor gene p53. However, p53 activates many other proteins, such as Puma, which is a regulator of apoptosis, or Mdm2, which is a negative regulator of p53 activity. Studies in a mouse model have demonstrated that the deletion of p21 can prolong tissue preservation and the lifespan of aged, telomerase deficient mice with dysfunctional telomeres without concomitantly increasing the risk of cancer, since other target genes of p53 remained unaffected and limited the survival of genetically unstable cells, e.g. Puma-induced apoptosis remained intact (Begus-Nahrmann Y. et al. (2009) Nat. Genet. 41(10): 1138-1143; Sperka T. et al. (2011) Nat. Cell. Biol. 14(1): 73-79). In contrast, an inhibition of p53 led to the development of chromosomal instability in tissue stem cells, which caused deficiencies of differentiation and increased tissue atrophy (Begus-Nahrmann Y. et al. (2009), supra). Therefore, in order to improve regeneration of aging tissues without inducing chromosomal instability it is of fundamental importance to inhibit the p53 network only very specifically. The studies in the mouse model constitute a proof of principle that improved regeneration could be achieved by selective inhibition of p21.

In addition to its role in aging, p21 is essential for the survival of certain tumor cells (e.g. leukemia cells), since these tumor cells increasingly accumulate chromosomal instabilities after knock-out of p21, which finally causes tumor cells to die (Viale A. et al. (2009) Nature, 457(7225): 51-56). In addition, the concept emerged that stearing p53 responses towards apoptosis rather than cell cycle arrest could increase the treatment success of chemotherapies to kill tumor cells in cancer treatment (Waldman T. et al. (1997) Nat Med, 3(9): 1034-1036). It is conceivable that the unspecific inhibition of p53 or of a larger number of target genes of p53 will not show the same antitumor effect as the specific inhibition of p21, since the former approaches might involve inhibition of apoptosis, which is required for the inhibition of tumor growth. In accordance with this hypothesis, there are a number of publications showing that loss of p53 is a tumor inducing event occurring in many human tumors, whereas a selective loss of p21 is hardly ever observed in human tumors (Shiohara M. et al. (1994) Blood, 84(11): 3781-3784).

In summary, studies on the mouse model and on human tissue biopsies and tumor biopsies suggest that a selective inhibition of p21 might have positive effects on the regeneration capability of tissues and might inhibit tumor growth. In addition, p21 inhibition could also ameliorate the increased secretion of cytokines and growth factors of cells carrying DNA damage or cells that are exposed to other types of stress. This increased secretion of signaling molecules has been described as the “senescence associated secretory phenotype (SASP)” and it was shown that SASP can contribute to organismal dysfunction during aging (Ju Z. et al. (2007) Nat Med, 13(6): 742-747) as well as to an increased cancer risk by stimulating growth of neighboring tumor cells (Kratolica A. et al. (2001) Proc Natl Acad Sci USA, 98(21): 12072-12077).

Park and co-workers studied inhibitors of p21 and found out that such inhibitors exhibit an anti-proliferative and pro-apoptotic activity on kidney carcinoma cell lines (Park S.-H. et al. (2008) Cancer Biol. Ther. 7(12): 2015-2022). Such p21 inhibitors were identified in a protein affinity assay. Accordingly, these p21 inhibitors exert their activity by inhibiting the interaction between the p21 protein and its target proteins. However, Park et al. did not examine the specificity of their inhibitors. Furthermore, these p21 inhibitors only exhibited an effect in cell culture experiments when used in concentrations of more than 100 μM. Such high concentrations often involve unspecific and undesired reactions; therefore, it is highly doubtful whether the compounds described by Park et al. could be usable as specific p21 inhibitors in a clinical setting.

### TECHNICAL PROBLEMS UNDERLYING THE PRESENT INVENTION AND THEIR SOLUTION

Thus, there was a need in the prior art for selective p21 inhibitors.

The inventors strenuously searched for such selective and potent p21 inhibitors and surprisingly identified several novel compounds that selectively inhibit p21 at concentrations in the nanomolar or low micromolar range. In particular, the p21 inhibitors of the present invention do not inhibit p53 or other genes targeted by p53. Furthermore, it is pointed out that p21 inhibitors described in the prior art (see Park et al., supra) inhibit protein-protein interactions between p21 and its target proteins, whereas the inhibitors of the present invention have a completely different mode of action. More specifically, the inhibitors described herein surprisingly cause a transcriptional inhibition of p21 production within a cell.

The above-described objects are solved and the advantages are achieved by the subject-matter of the enclosed independent claims. Preferred embodiments of the invention are included in the dependent claims as well as in the following description, examples and figures.

The above overview does not necessarily describe all problems solved by the present invention.

### SUMMARY OF THE INVENTION

In a first aspect the present invention relates to a compound selected from the group consisting of compounds according to formula 1:

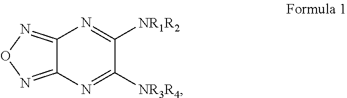

wherein
R₁ and R₂ are each independently selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or R₁ and R₂ together form a five-membered or six-membered heterocycloalkyl, heterocycloalkenyl or heteroaryl group, which is optionally substituted once, twice, or three times;
preferably R₁ is hydrogen and R₂ is selected from the group consisting of alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, preferably aryl or heteroaryl; wherein each group is optionally substituted once, twice, or three times,
in another preferred embodiment R₁ and R₂ are each independently selected from the group consisting of alkyl, heteroalkyl, haloalkyl, heterocycloalkyl, alkenyl, heteroalkenyl, alkynyl, and heteroalkynyl, preferably alkyl; wherein each group is optionally substituted once, twice, or three times;
R₃ and R₄ are each independently selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or R₃ and R₄ together form a five-membered or six-membered heterocycloalkyl, heterocycloalkenyl or heteroaryl group, which is optionally substituted once, twice, or three times
preferably R₃ is hydrogen and R₄ is selected from the group consisting of alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, preferably aryl or heteroaryl; wherein each group is optionally substituted once, twice, or three times;

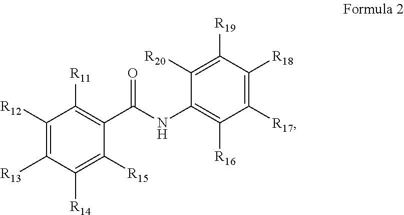

wherein
R₁₁, R₁₂, R₁₃, R₁₄, R₁₅, R₁₆, R₁₇, R₁₈, R₁₉ and R₂₀ are each independently selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times;
wherein
R^{I} is alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;

R^{II} and R^{III} are independently from each other selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or
R^{II} and R^{III} together form a five-membered or six-membered heterocycloalkyl, heterocycloalkenyl or heteroaryl group, wherein each group is optionally substituted once, twice, or three times;
R^{IV} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{V} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{VI} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;

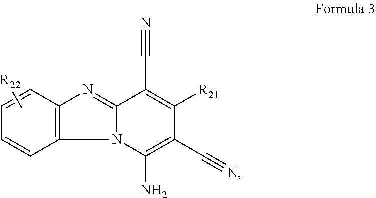

wherein
R₂₁ is selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₂₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R_{VI} are defined as above;

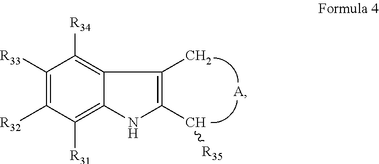

wherein
A is selected from the group consisting of —CH₂—, —CH₂—CH₂—, —CH₂—CH₂—CH₂—, and —CH₂—CH₂—CH₂—CH₂—, preferably —CH₂—CH₂— or —CH₂—CH₂—CH₂—CH₂—, more preferably —CH₂—CH₂—; optionally substituted once, twice, or three times by halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl; wherein R^{I}, R^{II}, R^{III}, R^{IV}, R^{V} and R^{VI} are defined as above;
R₃₁, R₃₂, R₃₃, and R₃₄ are each independently selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, preferably C₁ to C₅-alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above, preferably R₃₁, R₃₂, and R₃₃ are hydrogen and R₃₄ has the meaning as indicated above, preferably R₃₁, R₃₂, and R₃₄ are hydrogen and R₃₃ has the meaning as indicated above, preferably R₃₁, R₃₃, and R₃₄ are hydrogen and R₃₂ has the meaning as indicated above; preferably R₃₂, R₃₃, and R₃₄ are hydrogen and R₃₁ has the meaning as indicated above, most preferably R₃₁, R₃₂, and R₃₄ are hydrogen and R₃₃ has the meaning as indicated above;
R₃₅ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
preferably R₃₅ is —NR^{II}R^{III}; in this context it is prefered that R^{II} is hydrogen and R^{III} is selected from the group consisting of alkyl, preferably C₁ to C₅-alkly, —CO—NHR^{V} heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times;
wherein each group is optionally substituted once, twice, or three times; wherein R^{I}, R^{II}, R^{III}, R^{IV}, R^{V} and R^{VI} are defined as above;

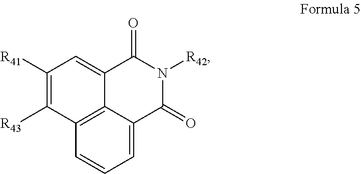

wherein
R₄₁ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R¹, R^{II}, R^{III} R^{IV}, R^{V} and R^{VI} are defined as above;
R₄₂ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;
R₄₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

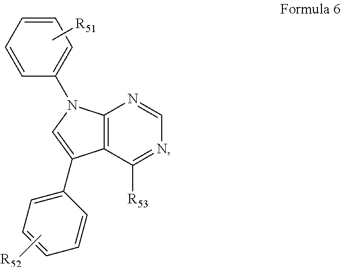

wherein
R₅₁ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₅₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₅₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

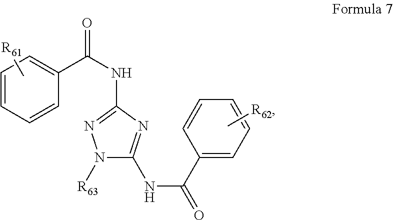

wherein
R₆₁ represents 0, 1, or 2 substituents that are independently selected from the group consisting of halogen, —OH, —OR^{I}, —SH, —NR^{II}R^{III}, —NO₂, —SO₂, —CO—OR^{VI}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₆₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₆₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

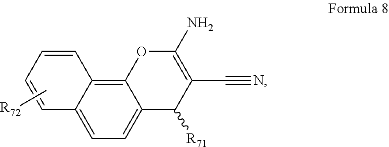

wherein
R₇₁ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;
R₇₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;

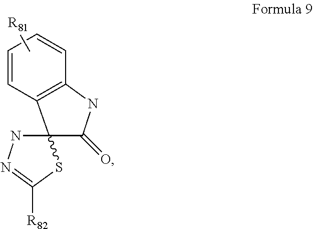

wherein
R₈₁ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R_{V} and R^{VI} are defined as above;

R₈₂ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

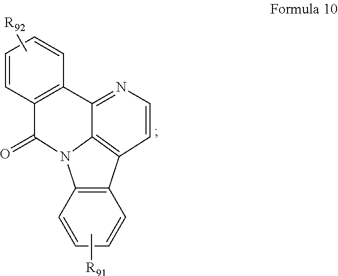

wherein
R₉₁ represents 0, 1, or 2 substituents that are independently selected from the group consisting of halogen, —OH, —OR^{I}, —SH, —NR^{II}R^{III}, —NOᵣ, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above; and
R₉₂ represents 0, 1, or 2 substituents that are independently selected from the group consisting of halogen, —OH, —OR¹, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, and R^{V} are defined as above; and pharmaceutically acceptable salts of any one of formulae 1 to 10.

In a second aspect the present invention relates to a compound according to the first aspect for use in medicine.

In a third aspect the present invention relates to a compound according to the first aspect for use in the treatment of cancer or for use in the induction of cell regeneration.

In a fourth aspect the present invention relates to a pharmaceutical composition comprising a compound according to the first aspect.

In a fifth aspect the present invention relates to an article of manufacture comprising: (a) a packaging material; (b) a compound according to the first aspect; and (c) a label or packaging insert contained within the packaging material indicating that patients receiving treatment with said compound can be treated for cancer and/or indicating that cell regeneration is induced in patients receiving treatment with said compound.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following paragraphs, definitions of the terms: alkyl, heteroalkyl, haloalkyl, cycloalkyl, heterocycloalkyl, alicyclic system, aryl, aralkyl, heteroaryl, heteroaralkyl, alkenyl, cycloalkenyl, heteroalkenyl, heterocycloalkenyl, and alkynyl are provided. These terms will in each instance of its use in the remainder of the specification have the respectively defined meaning and preferred meanings Nevertheless, in some instances of their use throughout the specification preferred meanings of these terms are indicated.

The term “alkyl” refers to a saturated straight or branched carbon chain. Preferably, the chain comprises from 1 to 10 carbon atoms, i.e. 1, 2, 3, 4, 5, 6, 7, 8, 9 or 10 e.g. methyl, ethyl propyl, iso-propyl, butyl, iso-butyl, sec-butyl, tert-butyl, pentyl, hexyl, heptyl, octyl, nonyl, decyl. Alkyl groups are optionally substituted.

The term “heteroalkyl” refers to a saturated straight or branched carbon chain. Preferably, the chain comprises from 1 to 9 carbon atoms, i.e. 1, 2, 3, 4, 5, 6, 7, 8, or 9, e.g. methyl, ethyl, propyl, iso-propyl, butyl, iso-butyl, sec-butyl, tert-butyl, pentyl, hexyl, heptyl, octyl, nonyl, which is interrupted one or more times, e.g. 1, 2, 3, 4, 5, with the same or different heteroatoms. Preferably, the heteroatoms are selected from O, S, and N, e.g. —(CH₂)ₙ—X—(CH₂)ₘCH₃, with n=0, 1, 2, 3, 4, 5, 6, 7, 8, or 9, m=0, 1, 2, 3, 4, 5, 6, 7, 8, or 9 and X═S, O or NR′ with R′═H or hydrocarbon (e.g. C₁ to C₆ alkyl). In particular, “heteroalkyl” refers to —O—CH₃, —OC₂H₅, —CH₂—O —CH₃, —CH₂—O—C₂H₅, —CH₂—O—C₃H₇, —CH_{2—O—C}₄H₉, —CH₂—O—C₅H₁₁, —C₂H₄—O—CH₃, —C₂H₄—O—C₂H₅, —C₂H₄-0—C₃H₇, —C₂H₄—O—C₄H₉ etc. Heteroalkyl groups are optionally substituted.

The term “haloalkyl” refers to a saturated straight or branched carbon chain in which one or more hydrogen atoms are replaced by halogen atoms, e.g. by fluorine, chlorine, bromine or iodine. Preferably, the chain comprises from 1 to 10 carbon atoms, i.e. 1, 2, 3, 4, 5, 6, 7, 8, 9 or 10. In particular, “haloalkyl” refers to —CH₂F, —CHF₂, —CF₃, —C₂H₄F, —C₂H₃F₂, —C₂H₂F₃, —C₂HF₄, —C₂F₅, —C₃H₆F, —C₃H₅F₂, —C₃H₄F₃, —C₃H₃F₄, —C₃H₂F₅, —C₃H₃F₆, —C₃F₇, —CH₂Cl, —CHCl₂, —CCl₃, —C₂H₄Cl, —C₂H₃Cl₂, —C₂H₂Cl₃, —C₂HCl₄, —C₂Cl₅, —C₃H₆Cl, —C₃H₅Cl₂, —C₃H₄Cl₃, —C₃H₃Cl₄, —C₃H₂Cl₅, —C₃HCl₆, and —C₃Cl₇. Haloalkyl groups are optionally substituted.

The terms “cycloalkyl” and “heterocycloalkyl”, by themselves or in combination with other terms, represent, unless otherwise stated, cyclic versions of “alkyl” and “heteroalkyl”, respectively, with preferably 3, 4, 5, 6, 7, 8, 9 or 10 atoms forming a ring, e.g. cyclopropyl, cyclobutyl, cyclopentyl, cyclohexyl, cycloheptyl, cyclooctyl etc. The terms “cycloalkyl” and “heterocycloalkyl” are also meant to include bicyclic, tricyclic and polycyclic versions thereof. If bicyclic, tricyclic or polycyclic rings are formed, it is preferred that the respective rings are connected to each other at two adjacent carbon atoms, however, alternatively the two rings are connected via the same carbon atom, i.e. they form a spiro ring system or they form “bridged” ring systems, preferably tricyclo[3.3.1.1^{3,7}]decan. The term “heterocycloalkyl” preferably refers to a saturated ring having five members of which at least one member is an N, O or S atom and which optionally contains one additional O or one additional N; a saturated ring having six members of which at least one member is an N, O or S atom and which optionally contains one additional O or one additional N or two additional N atoms; or a saturated bicyclic ring having nine or ten members of which at least one member is an N, O or S atom and which optionally contains one, two or three additional N atoms. “Cycloalkyl” and “heterocycloalkyl” groups are optionally substituted. Additionally, for heterocycloalkyl, a heteroatom can occupy the position at which the heterocycle is attached to the remainder of the molecule. Examples of cycloalkyl include cyclopropyl, cyclobutyl, cyclopentyl, cyclohexyl, cycloheptyl, spiro[3,3]heptyl, spiro[3,4]octyl, spiro[4,3]octyl, spiro[3,5]nonyl, spiro[5 ,3]nonyl, spiro[3,6]decyl, spiro[6,3]decyl, spiro[4,5]decyl, spiro[5,4]decyl, bicyclo[2.2.1]heptyl, bicyclo[2.2.2]octyl, adamantyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, 1,8-diazo-spiro[4,5]decyl, 1,7-diazo-spiro[4,5]decyl, 1,6-diazo-spiro[4,5]decyl, 2,8-diazo-spiro[4,5]decyl, 2,7-diazo-spiro[4,5]decyl, 2,6-diazo-spiro[4,5]decyl, 1,8-diazo-spiro[5,4]decyl, 1,7 diazo-spiro[5,4]decyl, 2,8-diazo-spiro[5,4]decyl, 2,7-diazo-spiro[5,4]decyl, 3 ,8-diazo-spiro[5,4]decyl, 3,7-diazo-spiro[5,4]decyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The term “alicyclic system” refers to mono, bicyclic, tricyclic or polycyclic version of a cycloalkyl or heterocycloalkyl comprising at least one double and/or triple bond. However, an alicyclic system is not aromatic or heteroaromatic, i.e. does not have a system of conjugated double bonds/free electron pairs. Thus, the number of double and/or triple bonds maximally allowed in an alicyclic system is determined by the number of ring atoms, e.g. in a ring system with up to 5 ring atoms an alicyclic system comprises up to one double bond, in a ring system with 6 ring atoms the alicyclic system comprises up to two double bonds. Thus, the “cycloalkenyl” as defined below is a preferred embodiment of an alicyclic ring system. Alicyclic systems are optionally substituted.

The term “aryl” preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphthyl or anthracenyl. The aryl group is optionally substituted.

The term “aralkyl” refers to an alkyl moiety, which is substituted by aryl, wherein alkyl and aryl have the meaning as outlined above. An example is the benzyl radical. Preferably, in this context the alkyl chain comprises from 1 to 8 carbon atoms, i.e. 1, 2, 3, 4, 5, 6, 7, or 8, e.g. methyl, ethyl, propyl, iso-propyl, butyl, iso-butyl, sec-butyl, tert-butyl, pentyl, hexyl, heptyl, octyl. The aralkyl group is optionally substituted at the alkyl and/or aryl part of the group. Preferably the aryl attached to the alkyl has the meaning phenyl, naphthyl or anthracenyl.

The term “heteroaryl” preferably refers to a five or six-membered aromatic monocyclic ring wherein at least one of the carbon atoms is replaced by 1, 2, 3, or 4 (for the five membered ring) or 1, 2, 3, 4, or 5 (for the six membered ring) of the same or different heteroatoms, preferably selected from O, N and S; an aromatic bicyclic ring system with 8 to 12 members wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system with 13 to 16 members wherein 1, 2, 3, 4, 5, or 6 carbon atoms of the 13, 14, 15, or 16 carbon atoms have been replaced with the same or different heteroatoms, preferably selected from O, N and S. Examples are furanyl, thiophenyl, oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, 2,3-benzodiazinyl, quinoxalinyl, quinazolinyl, quinolinyl, 1,2,3-benzotriazinyl, or 1,2,4-benzotriazinyl.

The term “heteroaralkyl” refers to an alkyl moiety, which is substituted by heteroaryl, wherein alkyl and heteroaryl have the meaning as outlined above. An example is the 2-alkylpyridinyl, 3-alkylpyridinyl, or 2-methylpyridinyl radical. Preferably, in this context the alkyl chain comprises from 1 to 8 carbon atoms, i.e. 1, 2, 3, 4, 5, 6, 7, or 8, e.g. methyl, ethyl, propyl, iso-propyl, butyl, iso-butyl, sec-butyl, tert-butyl, pentyl, hexyl, heptyl, octyl. The heteroaralkyl group is optionally substituted at the alkyl and/or heteroaryl part of the group. Preferably the heteroaryl attached to the alkyl has the meaning oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, 2,3-benzodiazinyl, quinolinyl, isoquinolinyl, quinoxalinyl, quinazolinyl, quinolinyl, 1,2,3-benzotriazinyl, or 1,2,4-benzotriazinyl.

The terms “alkenyl” and “cycloalkenyl” refer to olefinic unsaturated carbon atoms containing chains or rings with one or more double bonds. Examples are propenyl and cyclohexenyl. Preferably, the alkenyl chain comprises from 2 to 8 carbon atoms, i.e. 2, 3, 4, 5, 6, 7, or 8, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl, octenyl. Preferably the cycloalkenyl ring comprises from 3 to 8 carbon atoms, i.e. 3, 4, 5, 6, 7, or 8, e.g. 1-cyclopropenyl, 2-cyclopropenyl, 1-cyclobutenyl, 2-cyclobutenyl, 1-cyclopentenyl, 2-cyclopentenyl, 3-cyclopentenyl, 1-cyclohexenyl, 2-cyclohexenyl, 3-cyclohexenyl, cycloheptenyl, cyclooctenyl.

The terms “heteroalkenyl” and “heterocycloalkenyl” refer to unsaturated versions of “heteroalkyl” and “heterocycloalkyl”, respectively. Thus, the term “heteroalkenyl” refers to an unsaturated straight or branched carbon chain. Preferably, the chain comprises from 1 to 9 carbon atoms, i.e. 1, 2, 3, 4, 5, 6, 7, 8, 9, which is interrupted one or more times, e.g. 1, 2, 3, 4, 5, with the same or different heteroatoms. Preferably, the heteroatoms are selected from O, S, and N. In case that one or more of the interrupting heteroatoms is N, the N may be present as an —NR′— moiety, wherein R′ is hydrogen or hydrocarbon (e.g. C₁ to C₆ alkyl), or it may be present as an ═N— or —N═ group, i.e. the nitrogen atom can form a double bond to an adjacent C atom or to an adjacent, further N atom. “Heteroalkenyl” groups are optionally substituted. The term “heterocycloalkenyl” represents a cyclic version of “heteroalkenyl” with preferably 3, 4, 5, 6, 7, 8, 9 or 10 atoms forming a ring. The term “heterocycloalkenyl” is also meant to include bicyclic, tricyclic and polycyclic versions thereof. If bicyclic, tricyclic or polycyclic rings are formed, it is preferred that the respective rings are connected to each other at two adjacent atoms. These two adjacent atoms can both be carbon atoms; or one atom can be a carbon atom and the other one can be a heteroatom; or the two adjacent atoms can both be heteroatoms. However, alternatively the two rings are connected via the same carbon atom, i.e. they form a spiro ring system or they form “bridged” ring systems. The term “heterocycloalkenyl” preferably refers to an unsaturated ring having five members of which at least one member is an N, O or S atom and which optionally contains one additional O or one additional N; an unsaturated ring having six members of which at least one member is an N, O or S atom and which optionally contains one additional O or one additional N or two additional N atoms; or an unsaturated bicyclic ring having nine or ten members of which at least one member is an N, O or S atom and which optionally contains one, two or three additional N atoms. “Heterocycloalkenyl” groups are optionally substituted. Additionally, for heteroalkenyl and heterocycloalkenyl, a heteroatom can occupy the position at which the heterocycle is attached to the remainder of the molecule.

The term “alkynyl” refers to unsaturated carbon atoms containing chains or rings with one or more triple bonds. Preferably, the alkynyl chain comprises from 2 to 8 carbon atoms, i.e. 2, 3, 4, 5, 6, 7, or 8, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, heptynyl, octynyl.

The terms “heteroalkynyl”, “cycloalkynyl”, and “heterocycloalkynyl” refer to moieties that basically correspond to “heteroalkenyl”, “cycloalkenyl”, and “heterocycloalkenyl”, respectively, as defined above but differ from “heteroalkenyl”, “cycloalkenyl”, and “heterocycloalkenyl” in that at least one double bond is replaced by a triple bond.

In one embodiment, carbon atoms or hydrogen atoms in alkyl, cycloalkyl, aryl, aralkyl, alkenyl, cycloalkenyl, alkynyl radicals may be substituted independently from each other with one or more elements selected from the group consisting of O, S, N or with groups containing one ore more elements, i.e. 1, 2, 3, 4, 5, 6, or more selected from the group consisting of O, S, and N.

Embodiments include alkoxy, cycloalkoxy, aryloxy, aralkoxy, alkenyloxy, cycloalkenyloxy, alkynyloxy, alkylthio, cycloalkylthio, arylthio, aralkylthio, alkenylthio, cycloalkenylthio, alkynylthio, alkylamino, cycloalkylamino, arylamino, aralkylamino, alkenylamino, cycloalkenylamino, alkynylamino radicals.

Other embodiments include hydroxyalkyl, hydroxycycloalkyl, hydroxyaryl, hydroxyaralkyl, hydroxyalkenyl, hydroxycycloalkenyl, hydroxyalkynyl, mercaptoalkyl, mercaptocycloalkyl, mercaptoaryl, mercaptoaralkyl, mercaptoalkenyl, mercaptocycloalkenyl, mercaptoalkynyl, aminoalkyl, aminocycloalkyl, aminoaryl, aminoaralkyl, aminoalkenyl, aminocycloalkenyl, aminoalkynyl radicals.

In another embodiment, one or more hydrogen atoms, e.g. 1, 2, 3, 4, 5, 6, 7, or 8 hydrogen atoms in alkyl, heteroalkyl, cycloalkyl, heterocycloalkyl, alicyclic system, aryl, aralkyl, heteroaryl, heteroaralkyl, alkenyl, cycloalkenyl, heteroalkenyl, heterocycloalkenyl, alkynyl radicals may be substituted independently from each other with one ore more halogen atoms, e.g. Cl, F, or Br. One preferred radical is the trifluoromethyl radical.

If two or more radicals can be selected independently from each other, then the term “independently” means that the radicals may be the same or may be different.

The term “optionally substituted” in each instance if not further specified refers to halogen (in particular F, Cl, Br, or I), —NO₂, —CN, —OR′″, —NR′R″, —COOR′″, —CONR′R″, —NR′C OR″, —NR″COR′″, —NR′CONR′R″, —NR′SO₂E, —COR′″; —SO₂NR′R″, —OOCR′″, —CR′″R″″OH, —R′″OH, and -E;
R′ and R″ is each independently selected from the group consisting of hydrogen, alkyl, alkenyl, alkynyl, cycloalkyl, heterocycloalkyl, aryl, aralkyl, and heteroaryl or together form a heteroaryl, or heterocycloalkyl;
R′″ and R′″ is each independently selected from the group consisting of hydrogen, alkyl, alkenyl, alkynyl, cycloalkyl, heterocycloalkyl, alkoxy, aryl, aralkyl, heteroaryl, and —NR′R″;
E is selected from the group consisting of alkyl, alkenyl, alkynyl, cycloalkyl, alkoxy, alkoxyalkyl, heterocycloalkyl, an alicyclic system, aryl and heteroaryl; optionally substituted.

“Pharmaceutically acceptable” means approved by a regulatory agency of the Federal or a state government or listed in the U.S. Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans.

The term “pharmaceutically acceptable salt” refers to a salt of a compound of the present invention. Suitable pharmaceutically acceptable salts of the compound of the present invention include acid addition salts which may, for example, be formed by mixing a solution of a compound described herein or a derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide a compound of formula 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61. A prodrug is an active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson L.A. and Tunek A. (1988) Drug Metabolism Reviews 19(2): 165-194 and Bundgaard H. “Design of Prodrugs”, Elsevier Science Ltd. (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard H. et al. (1989) J. Med. Chem. 32(12): 2503-2507). Also, drugs containing an acidic NH group, such as imidazole, imide, indole and the like, have been masked with N-acyloxymethyl groups (Bundgaard H. “Design of Prodrugs”, Elsevier Science Ltd. (1985)). Hydroxy groups have been masked as esters and ethers. EP 0 039 051 A2 discloses Mannich-base hydroxamic acid prodrugs, their preparation and use.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, a “patient” means any mammal or bird that may benefit from a treatment with the compounds described herein. Preferably, a “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), or primates including chimpanzees and human beings. It is particularly preferred that the “patient” is a human being.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

### Embodiments of the Invention

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect the present invention is directed to compound selected from the group consisting of:

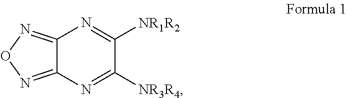

wherein
R₁ and R₂ are each independently selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or R₁ and R₂ together form a five-membered or six-membered heterocycloalkyl (e.g. morpholinyl), heterocycloalkenyl or heteroaryl group, which is optionally substituted once, twice, or three times;
preferably R₁ is hydrogen and R₂ is selected from the group consisting of alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, more preferably aryl or heteroaryl; wherein each group is optionally substituted once, twice, or three times,
in another preferred embodiment R₁ and R₂ are each independently selected from the group consisting of alkyl, heteroalkyl, haloalkyl, heterocycloalkyl, alkenyl, heteroalkenyl, alkynyl, and heteroalkynyl, preferably alkyl; wherein each group is optionally substituted once, twice, or three times;
R₃ and R₄ are each independently selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or R₃ and R₄ together form a five-membered or six-membered heterocycloalkyl (e.g. morpholinyl), heterocycloalkenyl or heteroaryl group, which is optionally substituted once, twice, or three times
preferably R₃ is hydrogen and R₄ is selected from the group consisting of alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, preferably aryl or heteroaryl; wherein each group is optionally substituted once, twice, or three times;

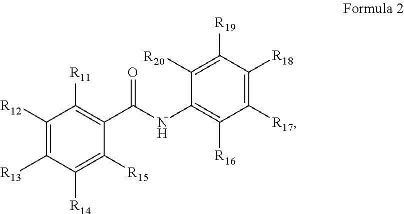

wherein
R₁₁, R₁₂, R₁₃, R₁₄, R₁₅, R₁₆, R₁₇, R₁₈, R₁₉ and R₂₀ are each independently selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times;
wherein
R^{I} is alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{II} and R^{III} are independently from each other selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or R^{II} and R^{III} together form a five-membered or six-membered heterocycloalkyl, heterocycloalkenyl or heteroaryl group, wherein each group is optionally substituted once, twice, or three times;
R^{IV} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{V} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;

R^{VI} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;

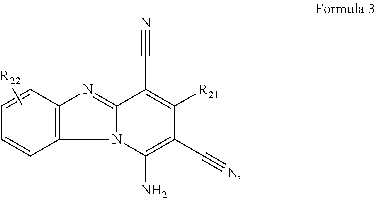

wherein
R₂₁ is selected from the group consisting of hydrogen, halogen, —OH, —OR^{I}, —SH, —NR^{II}R^{III}, —NO₂, —SO₂, —CO—O^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₂₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V}, and R^{VI} are defined as above;

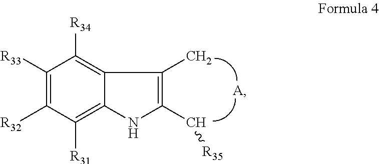

wherein
A is selected from the group consisting of —CH₂—, —CH₂—CH₂—, —CH₂—CH₂—CH₂—, and —CH₂—CH₂—CH₂—CH₂—, preferably —CH₂—CH₂— or —CH₂—CH₂—CH₂—CH₂-, more preferably —CH₂—CH₂—; optionally substituted once, twice, or three times by halogen, —OH, —OR^{I}, —SH, —NR^{II}R¹¹¹, —NO₂, —SO₂, —CO—OT^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl; wherein R^{I}, R^{II}, R^{III}, R^{IV}, R^{V} and R^{VI} are defined as above;

R₃₁, R₃₂, R₃₃, and R₃₄ are each independently selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, preferably C₁ to C₅-alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above, preferably R₃₁, R₃₂, and R₃₃ are hydrogen and R₃₄ has the meaning as indicated above, preferably R₃₁, R₃₂, and R₃₄ are hydrogen and R₃₃ has the meaning as indicated above, preferably R₃₁, R₃₃, and R₃₄ are hydrogen and R₃₂ has the meaning as indicated above; preferably R₃₂, R₃₃, and R₃₄ are hydrogen and R₃₁ has the meaning as indicated above, most preferably R₃₁, R₃₂, and R₃₄ are hydrogen and R₃₃ has the meaning as indicated above;
R₃₅ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
preferably R₃₅ is —NR^{II}R^{III}; in this context it is prefered that R^{II} is hydrogen and R^{III} is selected from the group consisting of alkyl, preferably C₁ to C₅-alkly, —CO—NHR^{V} heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times, a particular preferred meaning of R^{III} is C₁, C₂, C₃, C₄, or C₅-alkyl or heteroalkyl, which is substituted with a cycloalkyl, heterocycloalkly, aryl or heteroaryl.
wherein R^{I}, R^{II}, R^{III}, R^{IV}, R^{V} and R^{VI} are defined as above
in one preferred embodiment in which A is —CH₂—CH₂—CH₂—CH₂— R₃₅ is hydrogen,
in another preferred embodiment in which A is —CH₂—CH₂—R₃₅ is —NR^{II}R^{III}, in this context it is prefered that R^{II} is hydrogen and R^{III} is selected from the group consisting of alkyl, preferably C₁ to C₅-alkly, —CO—NHR^{V} heteroalkyl, haloalkyl, wherein each group is optionally substituted once, twice, or three times, preferably with —OH, aryl, heteroaryl, cycloalkyl or halogen, more preferably halogen or cycloalkyl;

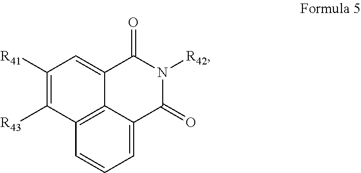

wherein
R₄₁ is hydrogen, halogen, —OH, —OR¹, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;
R₄₂ is hydrogen, halogen, —OH, —OR¹, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}R^{V} and R^{VI} are defined as above;
R₄₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

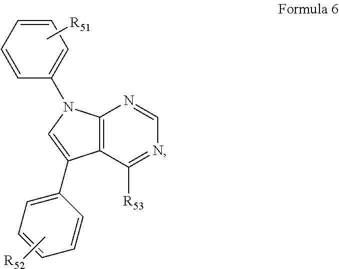

wherein
R₅₁ represents 0, 1, or 2 substituents (preferably 1 substituent) that are independently selected from the group consisting of halogen, —OH, —OR^{I}, —SH, ^{—NR}^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₅₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V}, R^{VI} are defined as above;
R₅₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

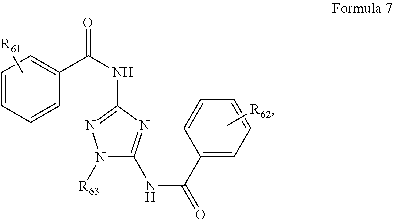

wherein
R₆₁ represents 0, 1, or 2 substituents (preferably 1 substituent) that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R¹, R^{II}, R^{III}, R^{IV}, R^{V} and R^{VI} are defined as above;
R₆₂ represents 0, 1, or 2 substituents (preferably 1 substituent) that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R_{VI} are defined as above;
R₆₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

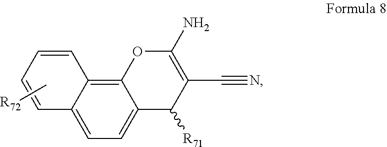

wherein
R₇₁ is hydrogen, halogen, —OH, —OR^{I}, —SH, —NR^{II}R^{III}, —NO₂, —SO₂, —CO—OR^{IV}, —CO—NHR^{V}, —SO₂, —NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;
R₇₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;

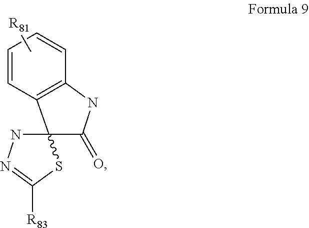

wherein
R₈₁ represents 0, 1, or 2 substituents (preferably 1 substituent) that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₈₂ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

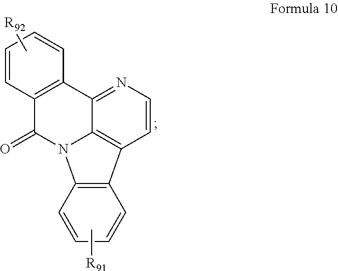

wherein
R₉₁ represents 0, 1, or 2 substituents that are independently selected from the group consisting of halogen, —OH, —OR^{I}, —SH, —NR^{II}R^{III}, —NO₂, —SO₂, —CO-OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, and R^{VI} are defined as above; and
R₉₂ represents 0, 1, or 2 substituents that are independently selected from the group consisting of halogen, —OH, —OR^{I}, —SH, —NR^{II}R^{III}, —NO₂, —SO₂, —CO-OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
and pharmaceutically acceptable salts of any one of formulae 1 to 10.

In preferred embodiments of the first aspect, both R₁ and R₃ are hydrogen. It is further preferred that at least one of R₂ and R₄ is aryl or heteroaryl (with aryl being particularly preferred), optionally substituted once, twice, or three times. In some embodiments, both R₂ and R₄ are aryl or heteroaryl (with aryl being particularly preferred), wherein each one of R₂ and R₄ is optionally substituted once, twice, or three times. In those embodiments, in which R₂ and/or R₄ are substituted, it is particularly preferred that such substitutents are selected from the group consisting of C₁ to C₆ alkyl (e.g. methyl, ethyl, propyl, butyl, pentyl, and hexyl), —CO—OR^{N}, —O—CO—R^{IV}, wherein R^{IV} is generally defined as above. In particularly preferred embodiments, though, R^{IV} is C₁ to C₆ alkyl (e.g. methyl, ethyl, propyl, butyl, pentyl, or hexyl).

In other preferred embodiments of the first aspect, both R₁ and R₂ are alkyl, preferably C₁ to C₆ alkyl (e.g. methyl, ethyl, propyl, butyl, pentyl, or hexyl).

In preferred embodiments of the first aspect, three or four of R₁₁, R₁₂, R₁₃, R₁₄, and R₁₅ are hydrogen, while one or two of R₁₁, R₁₂, R₁₃, R₁₄, and R₁₅ are independently selected from the group consisting of halogen, —OH, —OR^{I}, —SH, —N^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times. Preferably, one or two of R₁₁, R₁₂, R₁₃, R₁₄, and R₁₅ are independently selected from the group consisting of halogen, —OH, —OR^{I}, —CO—NHR^{V}, alkyl (e.g. methyl, ethyl, propyl, butyl, pentyl, or hexyl), and haloalkyl, wherein R^{I} and R^{V} are defined as above, and wherein each group is optionally substituted once, twice, or three times.

In further preferred embodiments of the first aspect, three or four of R₁₆, R₁₇, R₁₈, R₁₉ and R₂₀ are hydrogen, while one or two of R₁₆, R₁₇, R₁₈, R₁₉ and R₂₀ are independently selected from the group consisting of halogen, —OH, —OR^{I}, —SH, ^{—NR}^{II}RH^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times. Preferably, one or two of R₁₆, R₁₇, R₁₈, R₁₉ and R₂₀ are independently selected from the group consisting of halogen, —OH, —OR^{I}, —CO—NHR^{V}, alkyl (e.g. methyl, ethyl, propyl, butyl, pentyl, or hexyl), and haloalkyl, wherein R^{I} and R^{V} are defined as above, and wherein each group is optionally substituted once, twice, or three times.

In preferred embodiments of the first aspect, R₂₁ is selected from aryl and heteroaryl, wherein each group is optionally substituted once, twice, or three times. In further preferred embodiments, R₂₂ represents 0 substituents, i.e. R₂₂ is absent.

In preferred embodiments of the first aspect, A is —CH₂—CH₂— or —CH₂—CH₂—CH₂—CH₂—, most preferably A is —CH₂—CH₂—. In further preferred embodiments, R₃₂ and R₃₄ are hydrogen. It is additionally preferred that either R₃₁ or R₃₃ is hydrogen, while the other is selected from the group consisting of halogen, —OH, —OR^{I}, —SH, —NR^{II}R^{III}, —NO₂, —SO₂, —CO—OR^{IV}, —CO—NHR^{V}, —SP₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above. Preferably, one of R₃₁ and R₃₃ is hydrogen, while the other is selected from the group consisting of —SO₂—NHR^{VI} and alkyl (e.g. methyl, ethyl, propyl, butyl, pentyl, or hexyl), wherein R^{VI} is defined as above or is preferably aryl, optionally substituted once, twice, or three times.

In further preferred embodiments of the first aspect, R₃₅ is hydrogen or —NR^{II}R^{III}, (most preferably —NR^{II}R^{III}), wherein R^{II} and R^{III} are defined as above.

In preferred embodiments of the first aspect, R₄₁ is —NO₂, and/or R₄₃ is —NR^{II}R^{III}, wherein R^{II} and R^{III} are defined as above. In further preferred embodiments, R₄₂ is alkyl or heteroalkyl, optionally substituted once, twice, or three times.

In preferred embodiments of the first aspect, R₅₁ represents one substituent as defined above. More preferably, R₅₁ represents halogen (i.e. F, Cl, Br, or I). It is further preferred that R₅₂ represents 0 substituents, i.e. that R₅₂ is absent. In further preferred embodiments, R₅₃ is —NR^{II}R^{III}, wherein R^{II} and R^{III} are defined as above.

In preferred embodiments of the first aspect, R₆₁ represents one substituent as defined above. More preferably, R₆₁ represents halogen (i.e. F, Cl, Br, or I). It is further preferred that R₆₂ represents one substituent as defined above. More preferably, R₆₂ represents —OH or —OR^{I}, wherein R^{I} is defined as above. In further preferred embodiments, R₆₃ is aryl, optionally substituted once, twice, or three times.

In preferred embodiments of the first aspect, R₇₁ is aryl, optionally substituted once, twice, or three times. It is further preferred that R₇₂ represents 0 substituents, i.e. that R₇₂ is absent.

In preferred embodiments of the first aspect, R₈₁ represents 1 substituent as defined above. More preferably, R₈₁ represents alkyl (e.g. methyl, ethyl, propyl, butyl, pentyl or hexyl), optionally substituted once, twice, or three times. It is further preferred that R₈₂ is aryl, optionally substituted once, twice, or three times.

In preferred embodiments of the first aspect, R₉₁ represents 1 substituent as defined above. More preferably, R₉₁ represents —OH or —OR^{I}, wherein R^{I} is defined as above. Most preferably R₉₁ represents —OH. It is further preferred that R₉₂ represents 0 substituents, i.e. that R₉₂ is absent.

In preferred embodiments of the first aspect, the compound is selected from the group consisting of:

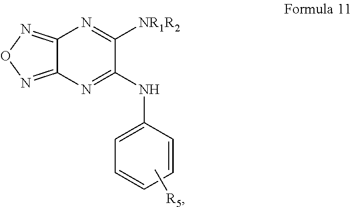

wherein
R₁ and R₂ are defined as shown above;
R₅ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as shown above
preferably R₅ is selected from alkyl, preferably C₁, C₂, C₃, C₄, or C₅-alkyl, more preferably methyl, —OR^{I} and —CO—OR^{IV};

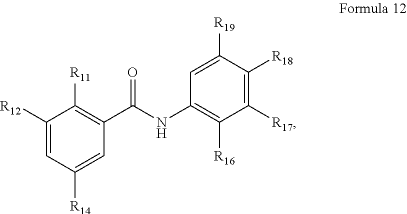

wherein R₁₁, R₁₂, R₁₄, R₁₆, R₁₇, R₁₈, and R₁₉ are defined as shown above;

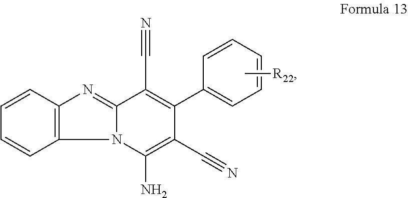

wherein R₂₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VIi}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as shown above;

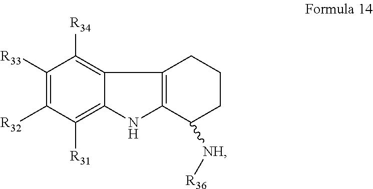

wherein
R₃₁, R₃₂, R₃₃, and R₃₄ are defined as shown above;
R₃₆ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, preferably C₁, C₂, C₃, C₄, or C₅, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above,
preferred substitutents R₃₆ are C₁, C₂, C₃, C₄, or C₅ alkyl or heteroalkyl, substituted with one or two, preferably one cycloalkyl, preferably C₅, C₆, C₇, C₈, C₉, C₁₀, C₁₁, C₁₂, C₁₃, C₁₄, or C₁₅, heterocycloalkyl, aryl or heteroaryl, preferably the ring(s) is(are) formed of between 5, 6, 7, 8, 9, 10, 11, 12, 13, 14 or 15 carbon and/or heteroatoms in total, i.e. counting carbon and, if present heteroatoms, particularly preferred are bicyclic or tricyclic ring systems, most preferably tricyclo[3.3.1.1^{3,7}]decan, the ring(s) are optionally substituted, preferably with 1, 2 or 3 substitutents, preferably selected from the group consisting of halogen, —OH, —OR^{I}, —SH, —NR^{II}R^{III}, —NO₂, —SO₂, and —CO—OR^{IV}
a preferred substituent of R₃₆ is —CO—NHR^{V}; wherein R^{V} preferably is hydrogen another preferred substituent of R₃₆ is aryl or heteroaryl, preferably phenyl; in each case optionally substituted once, twice, or three times, preferably with —OH, halogen, alkyl or alkoxy,

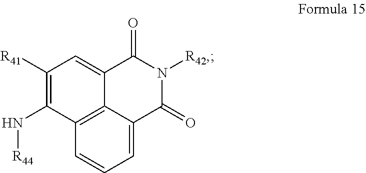

wherein
R₄₁ and R₄₂ are defined as shown above;
R₄₄ is hydrogen, halogen, —OH, —OR^{I}, —SH, —NR^{II}R^{III}, —NO₂, —SO₂, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above.

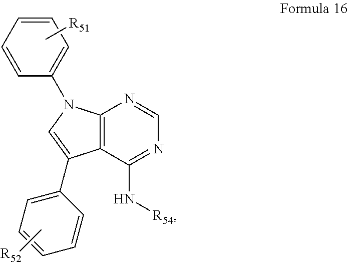

wherein
R₅₁ and R₅₂ are defined as shown above;
R₅₄ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;

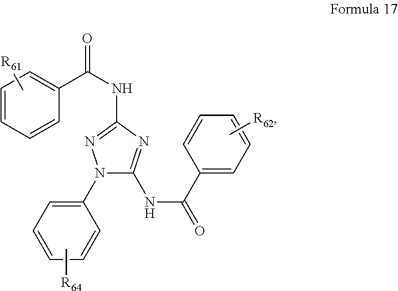

wherein
R₆₁ and R₆₂ are defined as shown above;
R₆₄ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as shown above;

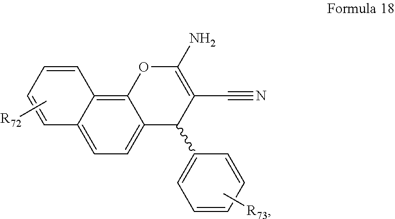

R₇₂ is defined as shown above;
R₇₃ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as shown above;

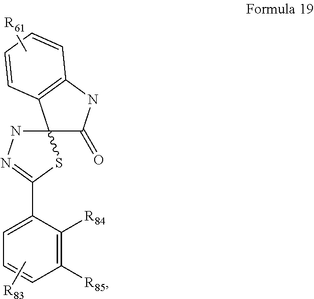

wherein
R₈₁ is defined as shown above;
R₈₃ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cyclo-alkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R_{VI} are defined as shown above;
R₈₄ and R₈₅ are each independently from the other selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as shown above; or R₈₄ and R₈₅ together form a five-membered or six-membered alkenyl, aryl, heterocycloalkenyl or heteroaryl group, which is condensed with the phenyl group to which R₈₄ and R₈₅ are attached and which is optionally substituted once, twice, or three times;

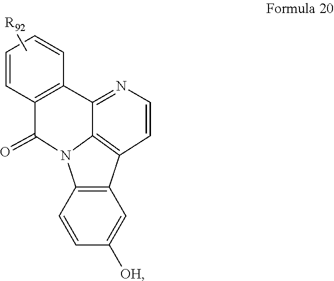

wherein R₉₂ is defined as shown above;
and pharmaceutically acceptable salts of any one of formulae 11 to 20.

In preferred embodiments of the first aspect, R₅ represents 1 or 2 substituents as defined above. It is further preferred that these 1 or 2 substituents are independently selected from the group consisting of —CO—OR^{IV} and alkyl (e.g. methyl, ethyl, propyl, butyl, pentyl, and hexyl), wherein R^{IV} is defined as shown above.

In preferred embodiments of the first aspect, R₂₂ represents 1 or 2 substituents as defined above. It is further preferred that these 1 or 2 substituents are independently selected from the group consisting of halogen, —OH, —OR^{I}, —NR^{II}R^{III}, and alkyl, wherein R^{I}, R^{II} and R^{III} are defined as shown above;

In preferred embodiments of the first aspect, R₃₆ is hydrogen, heteroalkyl, cycloalkyl, aryl, or aralkyl, wherein each group is optionally substituted once, twice, or three times.

In preferred embodiments of the first aspect, R₄₄ is aryl, optionally substituted once, twice, or three times.

In preferred embodiments of the first aspect, R₅₄ is aralkyl, optionally substituted once, twice, or three times.

In preferred embodiments of the first aspect, R₆₄ represents 0 substituents, i.e. R₆₄ is absent.

In preferred embodiments of the first aspect, R₇₃ represents 2 substituents that are independently selected from the group consisting of —OR^{I} and alkyl (e.g. methyl, ethyl, propyl, butyl, pentyl, and hexyl), wherein R^{I} is defined as shown above;

In preferred embodiments of the first aspect, R₈₃ represents 0 substituents, i.e. R₈₃ is absent. It is further preferred that R₈₄ and R₈₅ together form a five-membered or six-membered alkenyl, aryl, heterocycloalkenyl or heteroaryl group, which is condensed with the phenyl group to which R₈₄ and R₈₅ are attached and which is optionally substituted once, twice, or three times. Most preferably, R₈₄ and R₈₅ together form a six-membered aryl group (i.e. a phenyl group), which is condensed with the phenyl group to which R₈₄ and R₈₅ are attached (i.e. thereby forming a naphthyl group) and which is optionally substituted once, twice, or three times.

In particularly preferred embodiments of the first aspect, the compound is selected from the group consisting of:

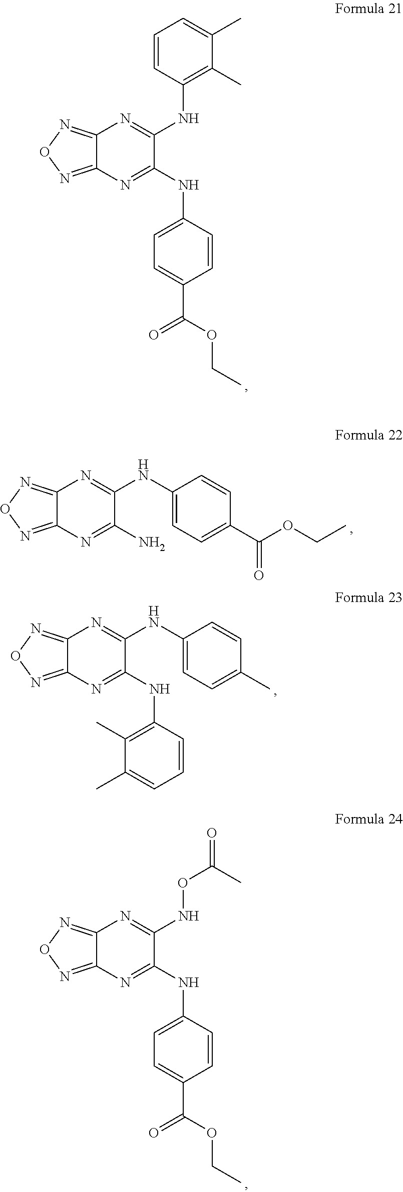 [SMILES: CCOC(=O)c1ccc(Nc2nc3nonc3nc2Nc2cccc(C)c2C)cc1 ; CCOC(=O)c1ccc(Nc2nc3nonc3nc2N)cc1 ; Cc1ccc(Nc2nc3nonc3nc2Nc2cccc(C)c2C)cc1 ; CCOC(=O)c1ccc(Nc2nc3nonc3nc2NOC(C)=O)cc1]

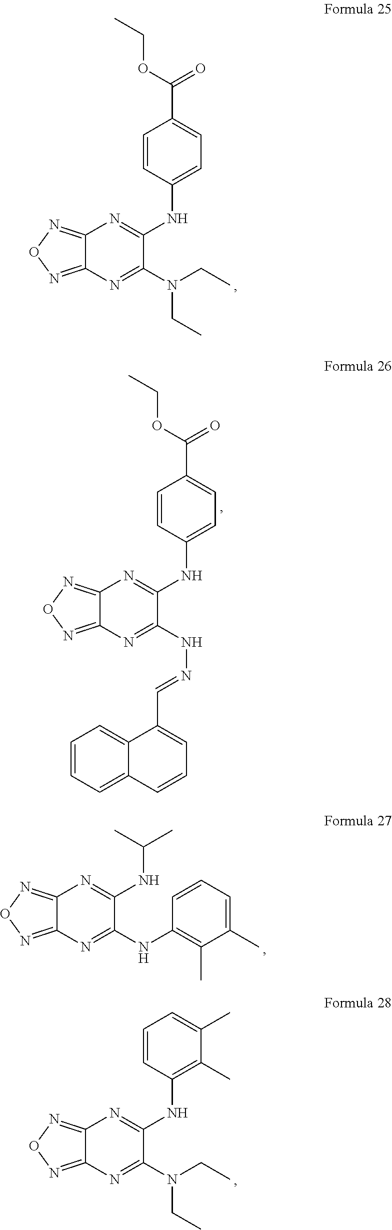

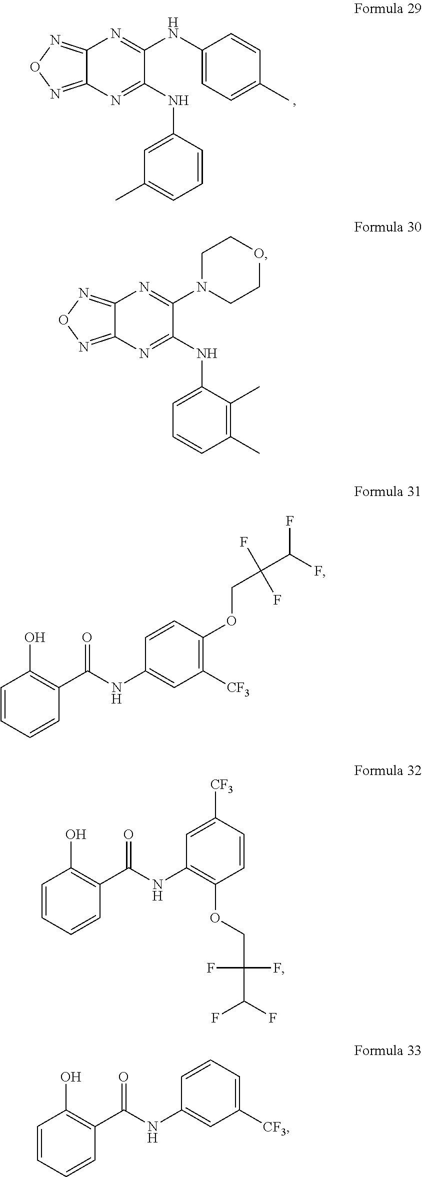

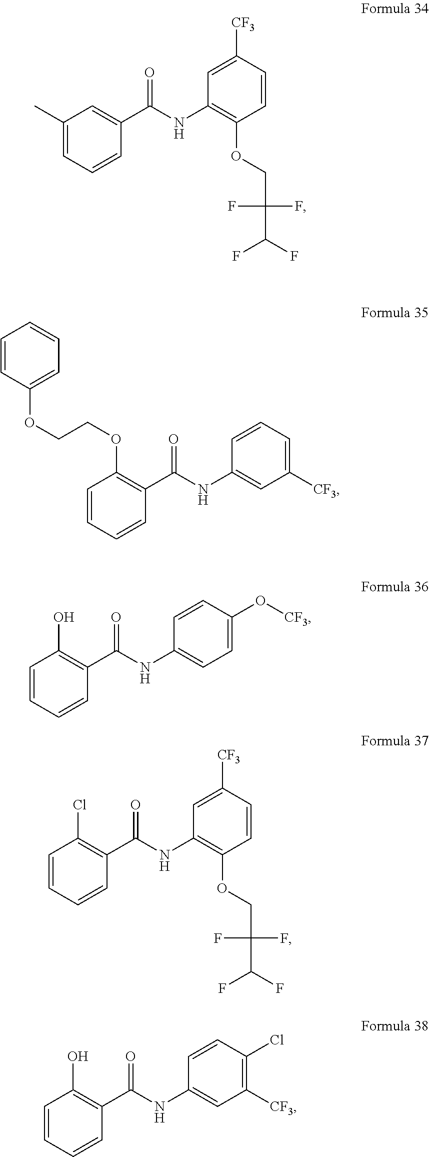 [SMILES: Cc1cccc(C(=O)Nc2cc(C(F)(F)F)ccc2OCC(F)(F)C(F)F)c1 ; O=C(Nc1cccc(C(F)(F)F)c1)c1ccccc1OCCOc1ccccc1 ; O=C(Nc1ccc(OC(F)(F)F)cc1)c1ccccc1O ; O=C(Nc1cc(C(F)(F)F)ccc1OCC(F)(F)C(F)F)c1ccccc1Cl ; O=C(Nc1ccc(Cl)c(C(F)(F)F)c1)c1ccccc1O]

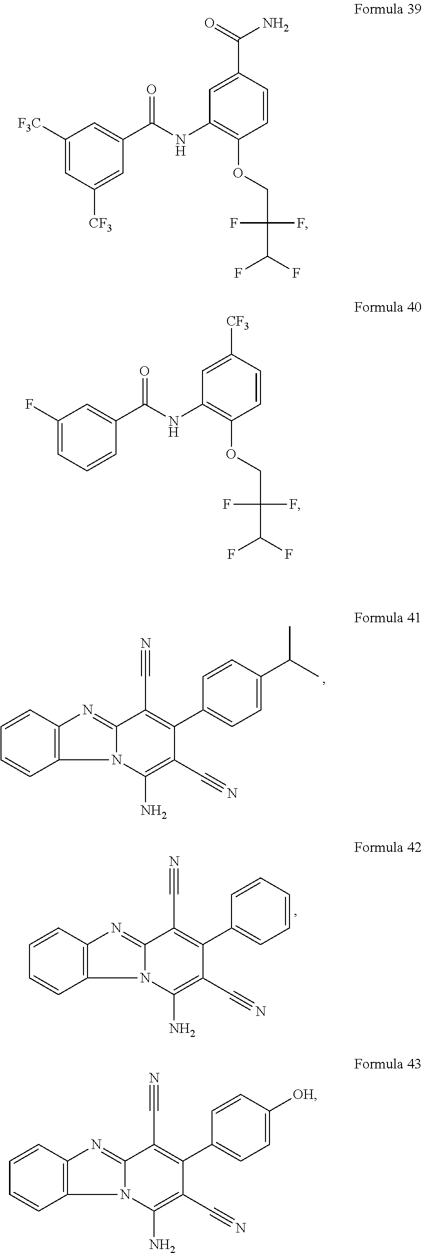 [SMILES: NC(=O)c1ccc(OCC(F)(F)C(F)F)c(NC(=O)c2cc(C(F)(F)F)cc(C(F)(F)F)c2)c1 ; O=C(Nc1cc(C(F)(F)F)ccc1OCC(F)(F)C(F)F)c1cccc(F)c1 ; CC(C)c1ccc(-c2c(C#N)c(N)n3c(nc4ccccc43)c2C#N)cc1 ; N#Cc1c(-c2ccccc2)c(C#N)c2nc3ccccc3n2c1N ; N#Cc1c(-c2ccc(O)cc2)c(C#N)c2nc3ccccc3n2c1N]

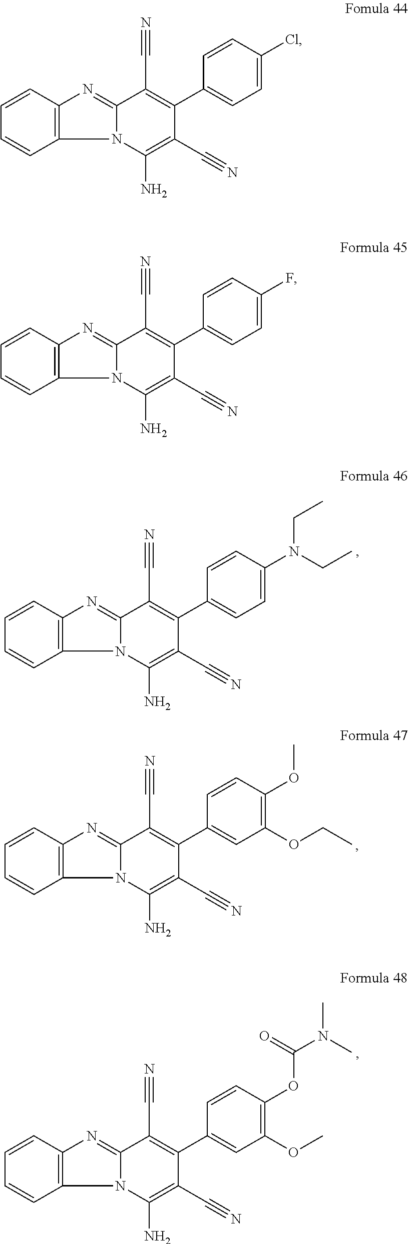 [SMILES: N#Cc1c(-c2ccc(Cl)cc2)c(C#N)c2nc3ccccc3n2c1N ; N#Cc1c(-c2ccc(F)cc2)c(C#N)c2nc3ccccc3n2c1N ; CCN(CC)c1ccc(-c2c(C#N)c(N)n3c(nc4ccccc43)c2C#N)cc1 ; CCOc1cc(-c2c(C#N)c(N)n3c(nc4ccccc43)c2C#N)ccc1OC ; COc1cc(-c2c(C#N)c(N)n3c(nc4ccccc43)c2C#N)ccc1OC(=O)N(C)C]

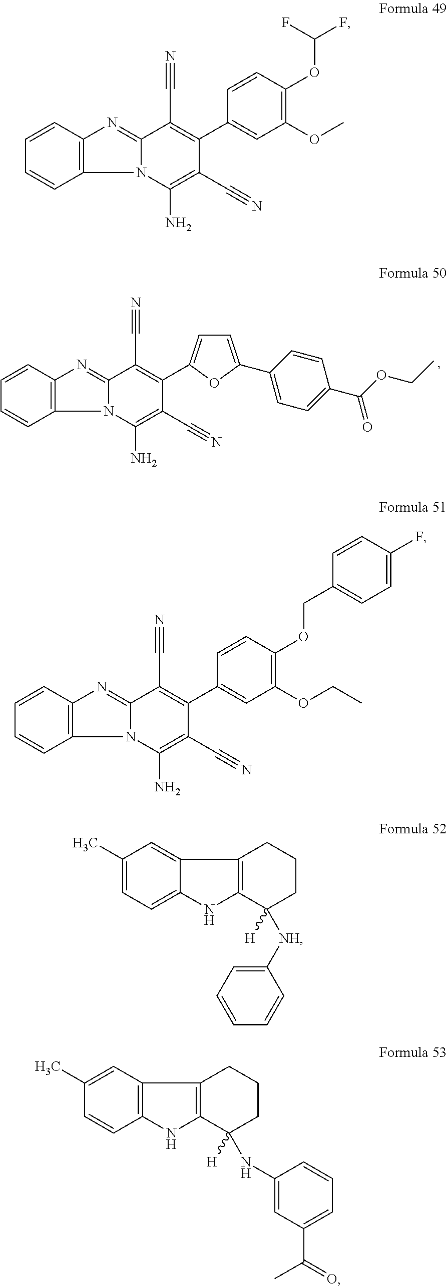

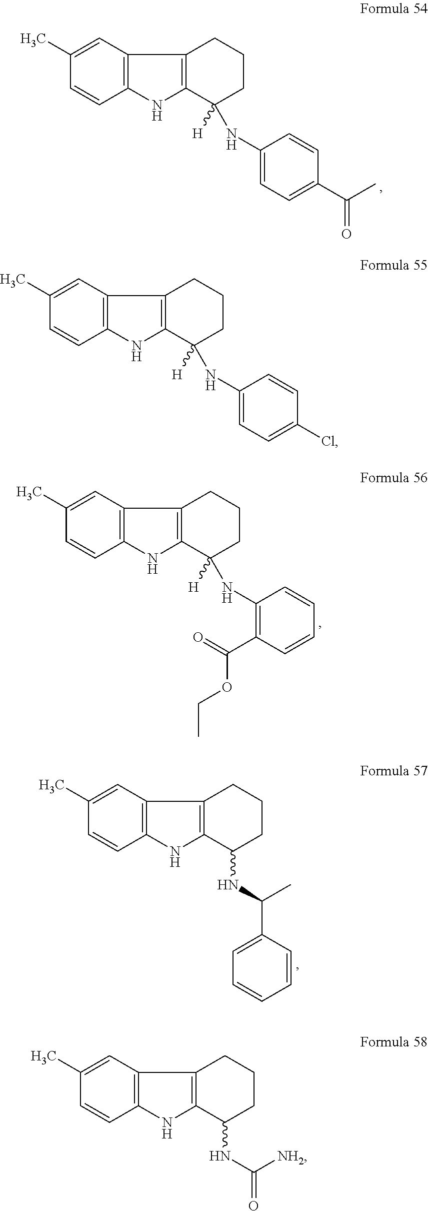

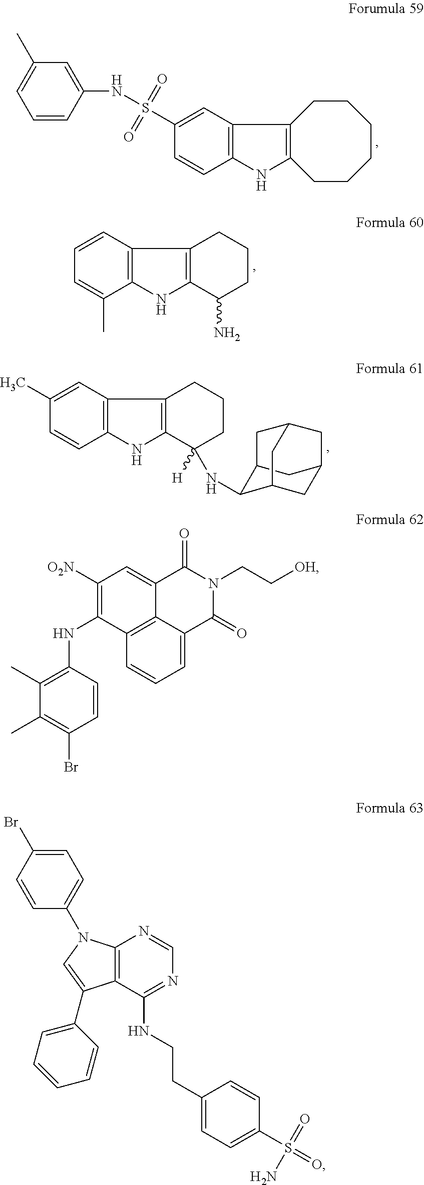 [SMILES: Cc1cccc(NS(=O)(=O)c2ccc3[nH]c4c(c3c2)CCCCCC4)c1 ; Cc1cccc2c3c([nH]c12)C(N)CCC3 ; Cc1ccc2[nH]c3c(c2c1)CCCC3NC1C2CC3CC(C2)CC1C3 ; Cc1c(Br)ccc(Nc2c([N+](=O)[O-])cc3c4c(cccc24)C(=O)N(CCO)C3=O)c1C ; NS(=O)(=O)c1ccc(CCNc2ncnc3c2c(-c2ccccc2)cn3-c2ccc(Br)cc2)cc1]

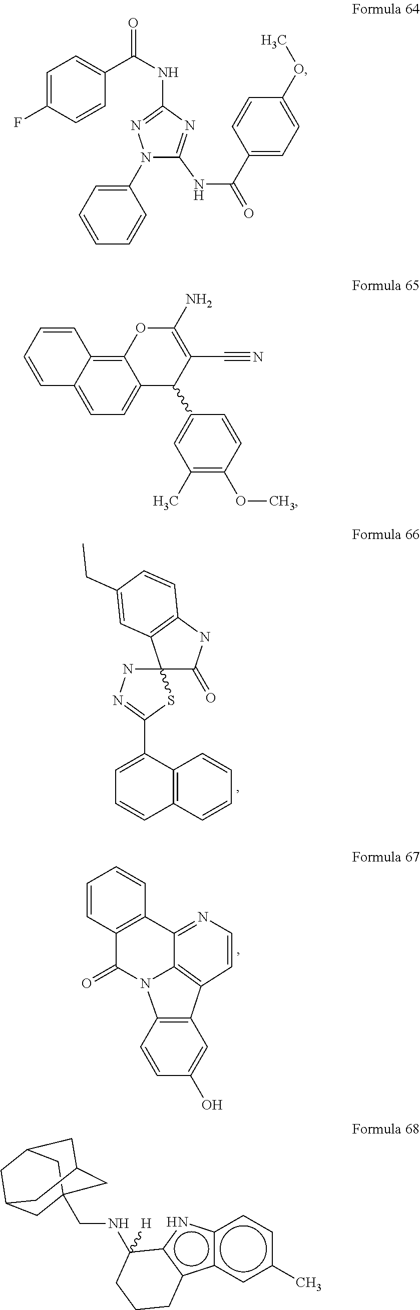

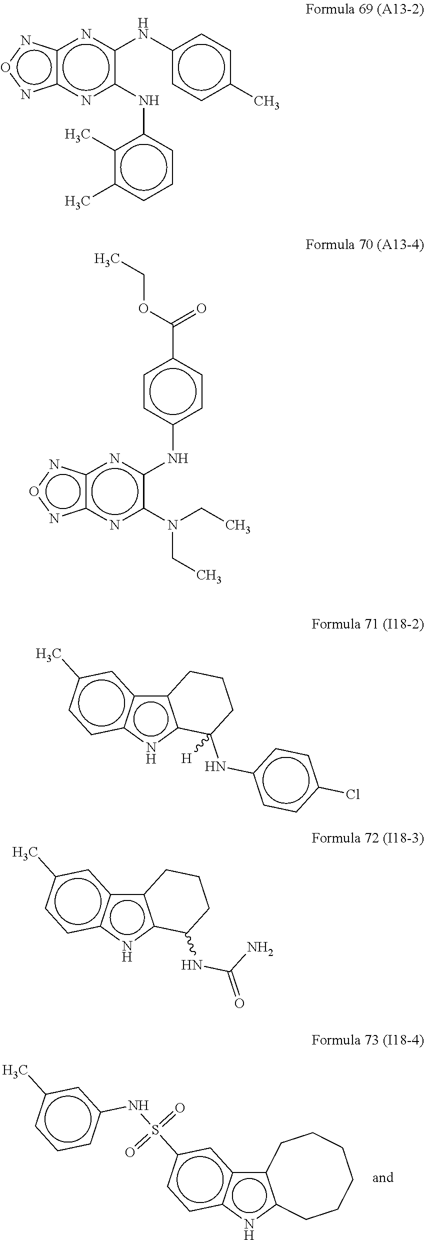

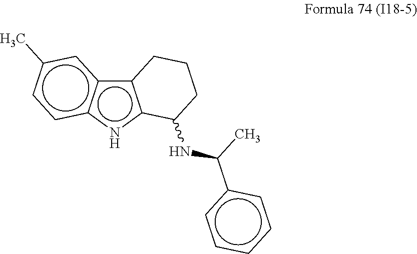

and pharmaceutically acceptable salts of any one of formulae 21 to 74.

In a second aspect the present invention is directed to a compound according to the first aspect for use in medicine.

In a third aspect the present invention is directed to a compound according to the first aspect for use in the treatment of cancer or for use in the induction of cell regeneration.

The third aspect of the present invention can alternatively be worded as follows: In a third aspect the present invention is directed to a method for treating cancer or for inducing cell regeneration in a subject, comprising the step: administering a therapeutic amount of the compound according to the first aspect to a subject in need thereof.

In preferred embodiments of the third aspect, the cancer is selected from the group consisting of breast cancer, kidney carcinoma, prostate cancer, cervical cancer, ovarian cancer, hepatocellular and squamous cell carcinoma, soft tissue sarcoma, leukemia (e.g. acute promyelocytic leukemia), glioma, and multiple myeloma. Treatment with p21-inhibitors might also be beneficial in other types of cancer.

In preferred embodiments of the third aspect, said compound is for use in the induction of cell regeneration in aging tissue, in tissue damaged by chronic diseases, or in tissue damaged by acute injury. Preferably, said induction of cell regeneration in aging tissue is used to ameliorate age-associated defects in muscle regeneration, sarcopenia, wound healing, cartilage atrophy, aging associated bone marrow failure, anemia, and others. Preferably, a said chronic disease is selected from the group consisting of liver cirrhosis, in particular liver cirrhosis as a consequence of chronic liver diseases, such as hepatitis or alcohol abuse, damages caused by chemotherapy, chronic HIV virus infection, bone marrow failure syndromes, chronic wounds, ulcerative colitis, and others. It is also preferred that said acute injury is selected from the group consisting of burns, lacerations, cuts, invasive surgical interventions, injuries caused by chemo- or radiotherapy, or any other type of tissue damage. Preferably, said tissue is selected from the group consisting of liver, skin, cartilage, bone marrow, intestine, muscle, and others.

In a fourth aspect the present invention is directed to a pharmaceutical composition comprising a compound according to the first aspect.

In preferred embodiments of the fourth aspect, the pharmaceutical composition further comprises one or more pharmaceutically acceptable diluents, carriers, excipients, fillers, binders, lubricants, glidants, disintegrants, adsorbents; and/or preservatives.

It is particularly preferred that the pharmaceutical composition of the fourth aspect can be used in the form of systemically or locally administered medicaments. These include parenterals, which comprise among others injectables and infusions. Injectables are formulated either in the form of ampoules or as so called ready-for-use injectables, e.g. ready-to-use syringes or single-use syringes and aside from this in puncturable flasks for multiple withdrawal. The administration of injectables can be in the form of subcutaneous (s.c.), intramuscular (i.m.), intravenous (i.v.), or intracutaneous (i.c.) application. Local applications include the local injection into joints to be used for induction of cartilage regeneration, or the topic treatment of skin to induce wound healing. In particular, it is possible to produce the respectively suitable injection formulations as a suspension of crystals, solutions, nanoparticular or a colloid dispersed systems like, e.g. hydrosols.

Injectable formulations can further be produced as concentrates, which can be dissolved or dispersed with aqueous isotonic diluents. The infusion can also be prepared in form of isotonic solutions, fatty emulsions, liposomal formulations and micro-emulsions. Similar to injectables, infusion formulations can also be prepared in the form of concentrates for dilution. Injectable formulations can also be applied in the form of permanent infusions both in in-patient and ambulant therapy, e.g. by way of mini-pumps.

It is possible to add to parenteral drug formulations, for example, albumin, plasma, expander, surface-active substances, organic diluents, pH-influencing substances, complexing substances or polymeric substances, in particular as substances to influence the adsorption of the compounds of the invention to proteins or polymers or they can also be added with the aim to reduce the adsorption of the compounds of the invention to materials like injection instruments or packaging-materials, for example, plastic or glass.

The compounds of the invention can be bound to microcarriers or nanoparticles in parenterals like, for example, to finely dispersed particles based on poly(meth)acrylates, polylactates, polyglycolates, polyamino acids or polyether urethanes. Parenteral formulations can also be modified as depot preparations, e.g. based on the “multiple unit principle”, if the compounds of the invention are introduced in finely dispersed, dispersed and suspended form, respectively, or as a suspension of crystals in the medicament or based on the “single unit principle” if a compound of the invention is enclosed in a formulation, e.g. in a tablet or a rod which is subsequently implanted. These implants or depot medicaments in single unit and multiple unit formulations often consist of so called biodegradable polymers like e.g. polyesters of lactic acid and glycolic acid, polyether urethanes, polyamino acids, poly(meth)acrylates or polysaccharides.

Adjuvants and carriers added during the production of the pharmaceutical compositions of the present invention formulated as parenterals are preferably aqua sterilisata (sterilized water), pH value influencing substances like, e.g. organic or inorganic acids or bases as well as salts thereof, buffering substances for adjusting pH values, substances for isotonization like e.g. sodium chloride, sodium hydrogen carbonate, glucose and fructose, tensides and surfactants, respectively, and emulsifiers like, e.g. partial esters of fatty acids of polyoxyethylene sorbitans (for example, Tween®) or, e.g. fatty acid esters of polyoxyethylenes (for example, Cremophor®), fatty oils like, e.g. peanut oil, soybean oil or castor oil, synthetic esters of fatty acids like, e.g. ethyl oleate, isopropyl myristate and neutral oil (for example, Miglyol®) as well as polymeric adjuvants like, e.g. gelatine, dextran, polyvinylpyrrolidone, additives which increase the solubility of organic solvents like, e.g. propylene glycol, ethanol, N,N-dimethylacetamide, propylene glycol or complex forming substances like, e.g. citrate and urea, preservatives like, e.g. benzoic acid hydroxypropyl ester and methyl ester, benzyl alcohol, antioxidants like e.g. sodium sulfite and stabilizers like e.g. EDTA.

When formulating the pharmaceutical compositions of the present invention as suspensions in a preferred embodiment thickening agents to prevent the setting of the compounds of the invention or, tensides and polyelectrolytes to assure the resuspendability of sediments and/or complex forming agents like, for example, EDTA are added. It is also possible to achieve complexes of the active ingredient with various polymers. Examples of such polymers are polyethylene glycol, polystyrol, carboxymethyl cellulose, Pluronics® or polyethylene glycol sorbit fatty acid ester. The compounds of the invention can also be incorporated in liquid formulations in the form of inclusion compounds e.g. with cyclodextrins. In particular embodiments dispersing agents can be added as further adjuvants. For the production of lyophilisates scaffolding agents like mannite, dextran, saccharose, human albumin, lactose, PVP or varieties of gelatine can be used.

In addition to local or systemic application the compounds can be used to generate enteral drugs that can be given orally to induce regeneration in aging or chronic disease. Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered via the nasal cavity to the lungs.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

Selection of the preferred effective dose will be determined by a skilled artisan based upon considering several factors which will be known to one of ordinary skill in the art. Such factors include the particular form of the pharmaceutic composition, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, according to standard clinical techniques.

In a fifth aspect the present invention is directed to an article of manufacture comprising: (a) a packaging material; (b) a compound according to the first aspect; and (c) a label or packaging insert contained within the packaging material indicating that patients receiving treatment with said compound can be treated for cancer and/or indicating that cell regeneration is induced in patients receiving treatment with said compound.

### FIGURES

FIG. 1a: Outline of a cell based screen to identify the p21 inhibitors. p53 expression was induced upon Doxycycline treatment, which leads to activation of downstream targets such as p21. Small organic compounds were added to cells to identify specific molecules which inhibit the expression of p21.

FIG. 1b: Identification of compounds with the desired activity. The ChemBioNet-collection, containing 29219 compounds, was screened. After the initial p21- and counter-screen against Mdm2, IC₅₀-validation and purity-analysis, 52 compounds were identified as specific inhibitors of the p53-mediated activation of the p21-inhibitor.

FIG. 2: Identification of target compounds which reduce p21 protein expression. Western Blot analysis with un-induced H1299 wt-p53 cells (C), Doxycycline-induced cells (Dox 05 μg/mL) and cells which were treated with the indicated inhibitors. The boxed inhibitors reduce p21-expression without affecting other proteins in the p53 pathway.

FIG. 3: p21-promotor activation in response to inhibitor treatment. Luciferase-assay with un-induced (C), Doxycycline-induced (Dox 0.5 μg/mL) and additionally added compounds (5 μM) H1299 wt-p53 p21P-Luc-5 cells (4 replicates with standard deviation).

FIG. 4: I-18 treatment improves wound healing in 24 week C57BL/6 mice Skin-biopsies (4-5 mm) were taken at Day 0 (D0) and lesions were either treated with 118 inhibitor (right panel) or DMSO treated as control (left panel). On day 2 and 4 (D2 and D4) the wound healing in the I18-treated mice was significantly improved when compared to DMSO treated control mice.

FIG. 5: Depicts a statistic evaluation of the wound healing area 0, 2, and 4 days after application of I18 or placebo to wound area. Skin biopsies (4-5 mm) were taken at Day 0 of young and old C57Bl6 mice and lesions were either treated with I18 inhibitor or DMSO. On day 2 the wound healing of old with I18-treated mice were significantly improved when compared to mock treated control mice of the same age. This trend further increased at Day 4.

FIG. 6: Decreased p21-mRNA expression in different organs of model organism Nothobranchius furzeri in response to I18-inhibitor treatment. p21-mRNA expression in different organs was analysed by quantitative PCR (qPCR) in response to treatment with the p21-inhibitor I18. Upper panel depicts the fold expression difference of p21-mRNA relative to tbp (TATA-box binding protein). Lower panel depicts fold expression of p21-mRNA relative to control (DMSO-treated animals). Note that there is an over 2-fold reduction in all tissues tested indicating the efficiency of p21-inhibitors across different species.

FIG. 7: The effect of preferred compounds of the invention in NIH 3T3 (Panel A) and BJ cells (Panel B). The inhibition of p21 expression is determined by Western blots with p(Ser15)p53, p53, Puma-α and p21 specific antibodies. A Western blot using β-actin specific antibodies is used as control in NIH 3T3 cells. The lower part shows a Comassie stain of the SDS-polyacrylamide prior to Western blotting.

FIG. 8: Assessment of p21 expression inhibitory activity of derivative I18-6 in comparison to I18 in HCT116 cells (human colon carcinoma cells). (A) I18-6 shows a notably improved inhibitory activity as indicated by Western blot. (B) Chemical structure of both compounds: I18 (left) and I18-6 (right).

FIG. 9: p21-inhibitors prevent binding of p53 to p21 promotor sequence. HCT119 cell lines were irradiated (6 Gy) and either mock treated (DMSO) or treated with different p21-inhibitors. 24 h post-irradiation ChIP was performed using anti p-p53 antibody and the promotor sequence of p21 was amplified with gene specific primers. Note that after treatment with p21-inhibitors p53 no longer binds to the p21-promotor region as indicated by loss of the 196 bp band (arrow). IP: Immuno-Precipitation, gDNA: genomic DNA.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Example 1

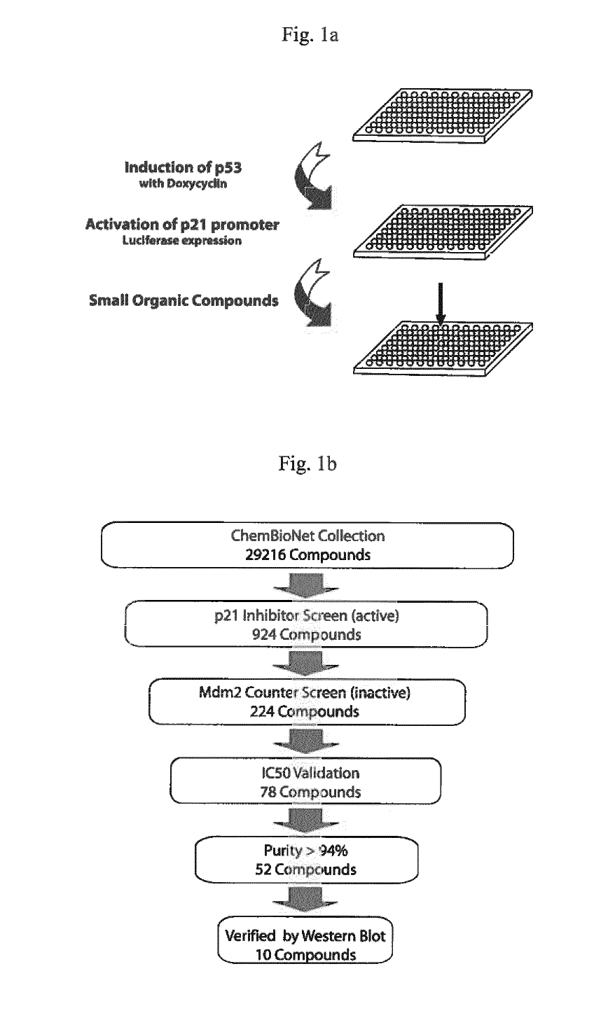

A cell-based screen was carried out to identify p21 inhibitors (FIG. 1a). H1299 cells (a cell line having a deletion in the p53 locus) were used. The H1299 cells contain a TetOn system for the inducible expression of p53. In addition, they contain luciferase under the control of the p21 promoter, which is activated by p53. Intracellular p53 expression was induced with doxycycline (0.5 μg/ml). Immediately thereafter, cells were exposed to test substances (5 μM) of the ChemBioNet collection (Leibniz Institut far Molekulare Pharmakologie, Berlin). About 20 hours later, cells were lysed and luciferase activity was determined.

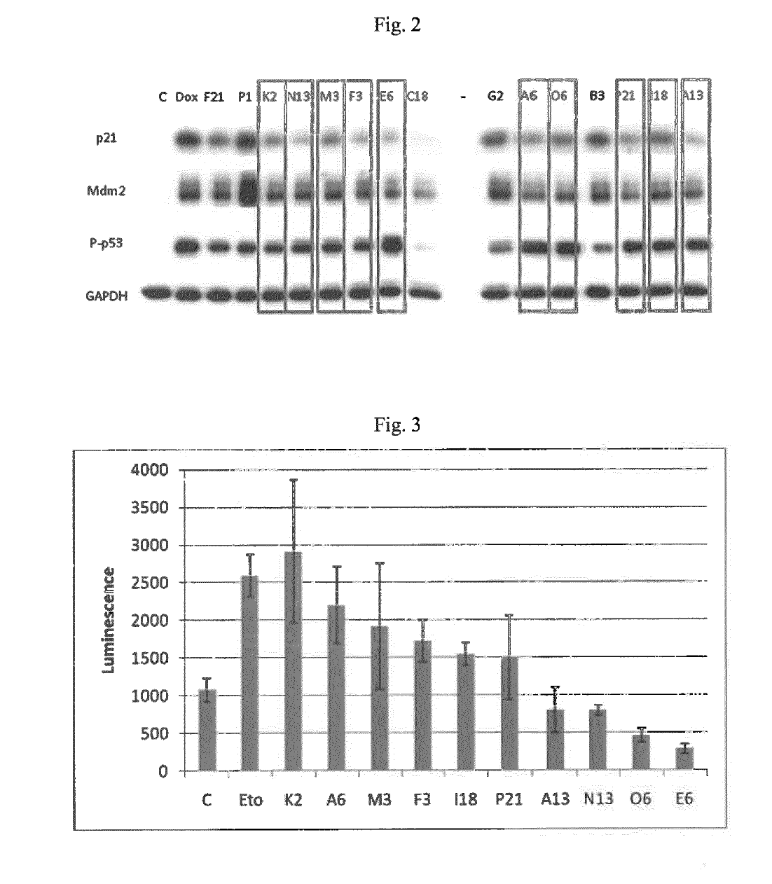

The counter-screen was carried out in the same way with another reporter cell line that contained luciferase under the control of the Mdm2 promoter. In this counter-screen, only those substances were examined which showed a z-score of <−3 in the p21 screen. Substances exhibiting a ratio in the relative activities of Mdm2 to p21 of at least 2, while not inhibiting Mdm2 more than 50%, were subjected to IC₅₀ validation. Substances having a purity of more than 94% were subsequently verified on the protein level by Western blot analysis (FIG. 2).

From the 29216 substances in the ChemBioNet collection, 10 substances were thus identified as specific inhibitors of p21 (FIG. 1b). These 10 substances were tested in a second cell line (HCT116-p21pLuc). This cell line also contains luciferase under control of the p21 promoter. DNA double strand breaks were induced in cells by exposition to etoposide (25 μM). Directly thereafter, cells were treated with the substances (5 μM). After 16 hours, luciferase activity was determined. Also in this experiment, most substances exhibited an inhibitory effect on the activation of p21 (FIG. 3).

IUPAC names of the 14 p21 inhibitors identified in example 1 are shown in Table 1 and are assigned to the abbreviations and formula numbers used in this specification.

TABLE 1
IUPAC names of compounds identified in example 1 and their IC₅₀ values.

| | Formula | | IC₅₀ |
|---|---|---|---|
| Abbreviation | number | IUPAC name | [μM] |
| A13 | 21 | Ethyl-4-[[5-(2,3-dimethylanilino)-[1,2,5]oxadiazolo[3,4-b]pyrazin-6- | 0.76 |
| | | yl]amino]benzoate | |
| K2 | 31 | 2-Hydroxy-N-[4-(2,2,3,3-tetrafluoropropoxy)-3- | 1.72 |
| | | (trifluoromethyl)phenyl)benzamide | |
| N13 | 41 | 1-Amino-3-(4-propan-2-ylphenyl)pyrido[1,2-a]benzimidazole-2,4- | 1.11 |
| | | dicarbonitrile | |
| M3 | 62 | 6-(4-Bromo-2,3-dimethylanilino)-2-(2-hydroxyethyl)-5- | 3.58 |
| | | nitrobenzo[de]isoquinoline-1,3-dione | |
| F3 | 63 | 4-[2-[[7-(4-Bromophenyl)-5-phenylpyrrolo[2,3-d]pyrimidin-4- | 6.08 |
| | | yl]amino]ethyl]benzenesulfonamide | |
| E6 | 64 | N-[5-[(4-Fluorobenzoyl)amino]-2-phenyl-1,2,4-triazol-3-y1]-4- | 0.49 |
| | | methoxybenzamide | |
| A6 | 65 | 2-amino-4-(4-methoxy-3-methylphenyl)-4H-benzo[h]chromene-3- | 1.13 |
| | | carbonitrile | |
| O6 | 66 | 5-Ethyl-5′naphthalen-1-ylspiro[1H-indole-3,2′-3H-1,3,4-thiadiazole]- | 3.68 |
| | | 2-one | |
| I18 | 52 | 6-methyl-N-phenyl-2,3,4,9-tetrahydro-1H-carbazol-1-amine | 0.17 |
| P21 | 67 | 5-hydroxy-9H-benzo[c]indolo[3,2,1-ij][1,5]naphthyridin-9-one | 1.07 |
| A13-2 | 69 | N5-(2,3-dimethylpheny1)-N6-(p-tolyl)-[1,2,5]oxadiazolo[3,4- | n.a. |
| | | b]pyrazine-5,6-diamine | |
| A13-4 | 70 | Ethyl 4-((6-pentan-3-ylamino)-[1,2,5]oxadiazolo[3,4-b]pyrazine-5- | n.a. |
| | | yl)amino)benzoate | |
| I18-2 | 71 | N-(4-chlorophenyl)-6-methyl-2,3,4,9-tetrahydro-1H-carbazol-1-amine | n.a. |
| I18-3 | 72 | 1-(6-methyl-2,3,4,9-tetrahydro-1H-carbazol-1-yl)urea | n.a. |
| I18-4 | 73 | N-(m-tolyl)-6,7,8,9,10,11-hexahydro-5H-cyclooctal[b]indole-2- | n.a. |
| | | sulfonamide | |
| I18-5 | 74 | 6-methyl-N-((S)-1-phenylethyl)-2,3,4,9-tetrahydro-1H-carbazol-1- | n.a. |
| | | amine | |
| I18-6 | 68 | | n.a. |

n.a., not available

### Example 2

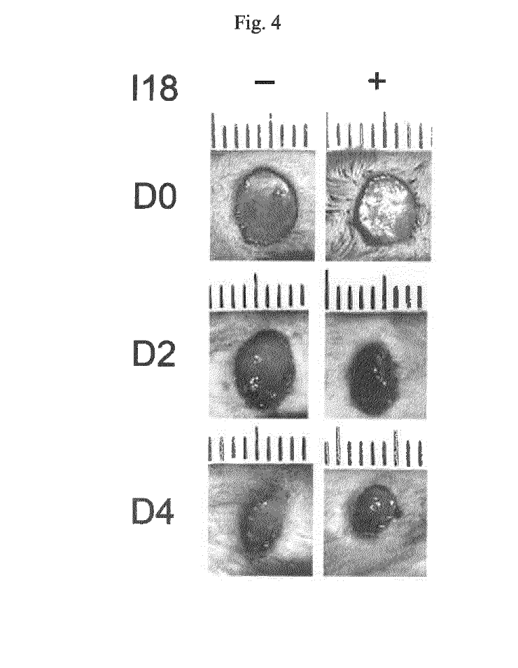

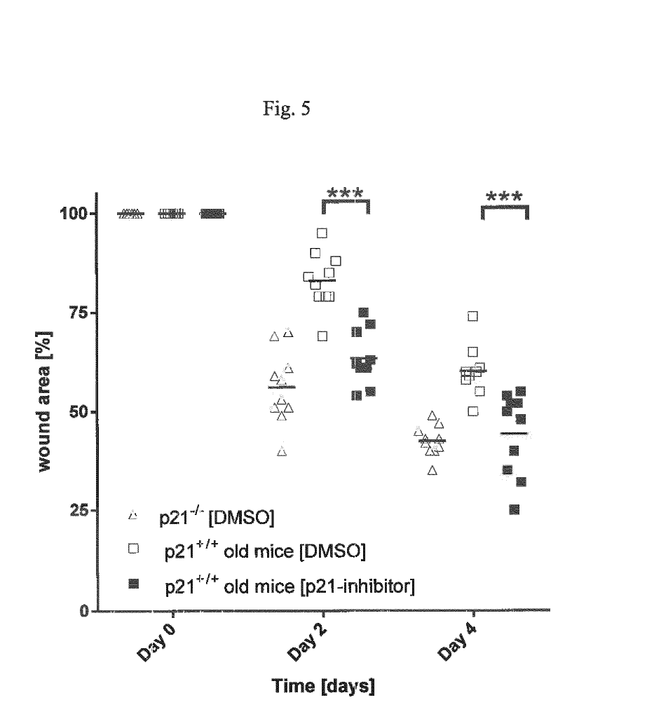

Skin-biopsies (4-5 mm) were taken at Day 0 (D0) and lesions were either treated with I18 compound (FIG. 4, right panel) or DMSO as control (FIG. 4, left panel) by intradermal injection [50 μM]. On day 2 and 4 (D2 and D4) the wound healing in the I18-treated mice was significantly improved when compared to control mice (FIG. 4 and FIG. 5). After 4 days the wound healing in old I18-treated mice was comparable to p21-deficient mice (FIG. 5), indicating that the improved wound healing was due to reduced p21-expression.

### Example 3

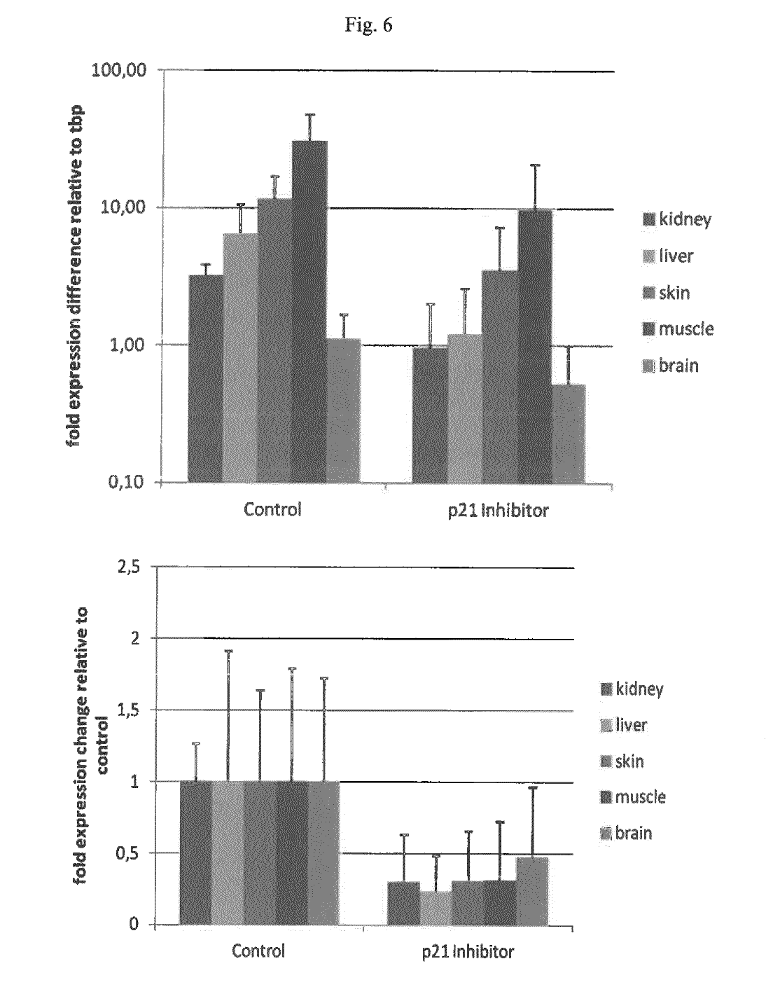

10 weeks old Nothobranchius furzeri were either injected intraperitoneal with I18-compound [10 μM] or with DMSO as control and sacrificed 48 h post treatment. RNA of various tissues was harvested and p21-mRNA expression was measured using quantitative PCR (qPCR). Fold expression was calculated relative to house-keeping gene tbp (TATA-box binding protein) (FIG. 6, upper panel) or control animals (FIG. 6, lower panel).

### Example 4

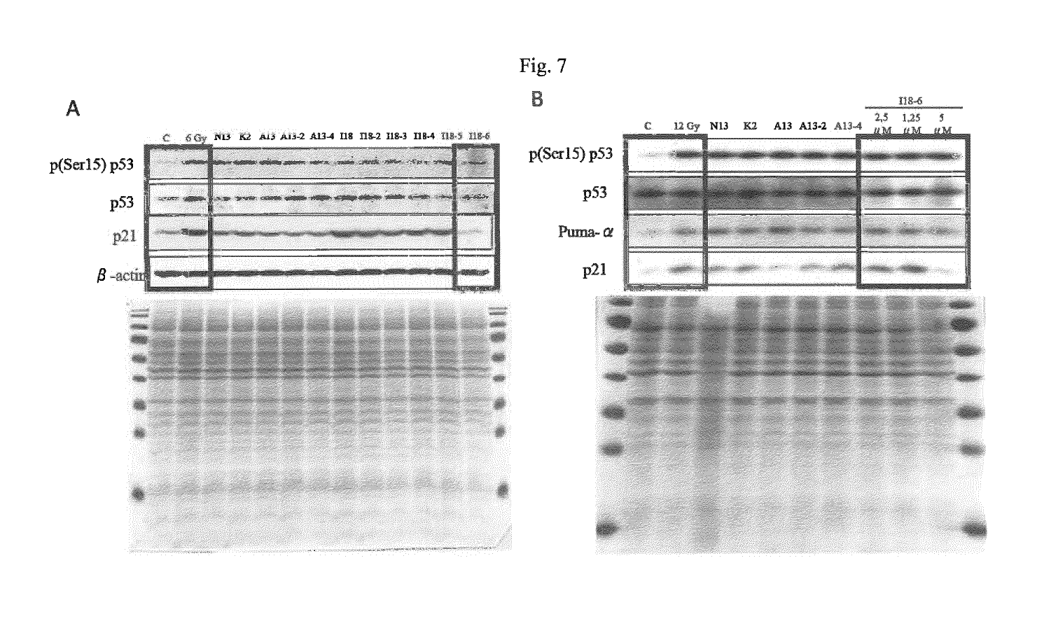

To test the specificity of a variety of different derivatives of the p21-inhibitor compound, two different cell lines (NIH3T3 and BJ cells) were either untreated (C) or irradiated with 6 Gy to induce p21 expression and subsequently treated with different derivatives of the compound (FIG. 7, left panel). In addition to p21-protein expression, expression of p(Ser15)-p53, p53 and Puma-α was tested. Only compounds reducing p21-expression but leaving other targets of the p53 signal pathway unaltered were considered for further testing.

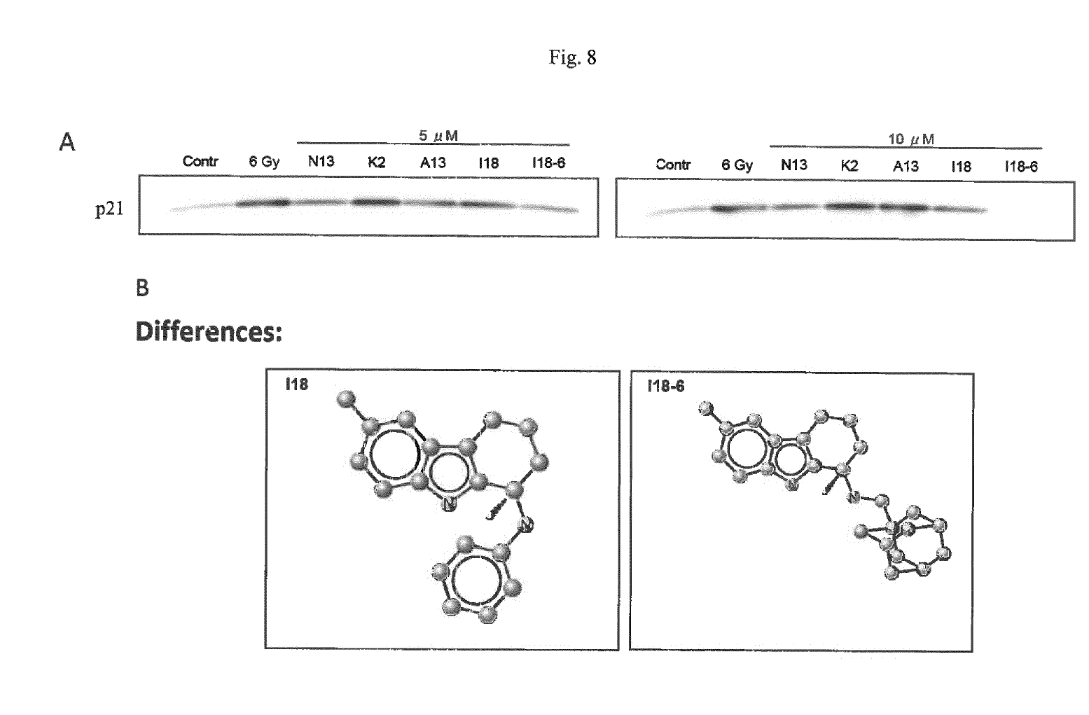

The most promising candidates were further tested for p(Ser15)p53, p53 and Puma (FIG. 7, right panel). Due to its specificity, the 118-6 derivate was also tested, using different concentrations (1.25-10 μM) (FIG. 7, right panel and FIG. 8A).

### Example 5

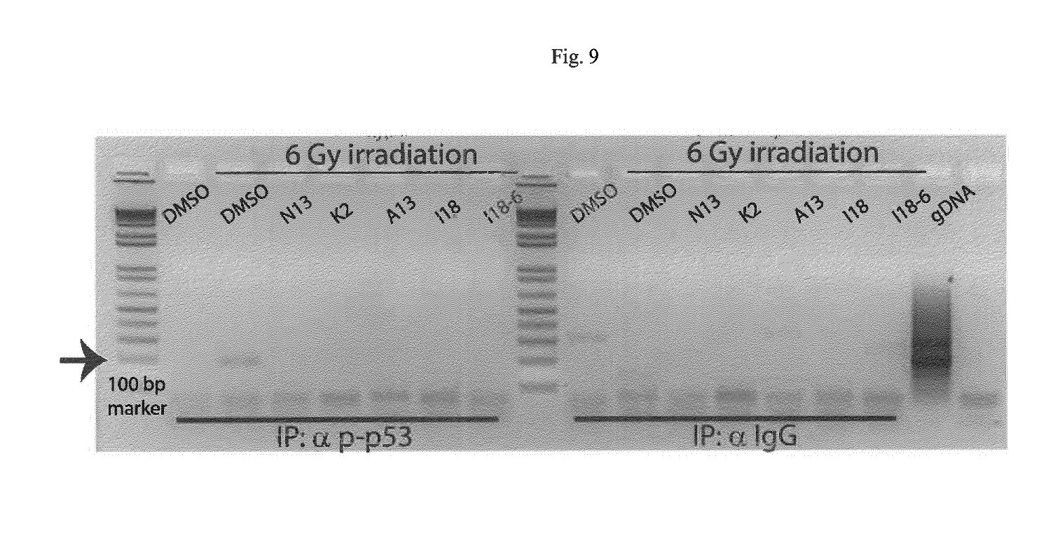

p21-Inhibitors prevent binding of p53 to the p21 promotor sequence in HCT119 p21+/+ cells. 2 hours prior to irradiation (6Gy), cells were either mock treated with DMSO (control) or treated with different p21-inhibitors (FIG. 9). 24 h post irradiation cells were harvested, proteins and DNA cross-linked and chromatin was immune-precipitated using p53 antibody. After elution of the chromatin from the antibody beads, DNA was purified and quantified by PCR. In the absence of p21-inhibitors, p53 binds specifically to the p21-promotor which could be detected by the presence of a 196 bp band in the irradiated control (see arrow). However, upon p21-inhibitor treatment the signal disappears, indicating the inability of p53 to bind to the p21-promotor. In addition, chromatin was also immunoprecipitated using antibody against IgG to exclude unspecific binding signals.

1. A compound selected from the group consisting of: 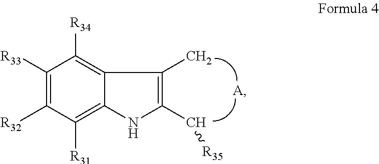
wherein
A is selected from the group consisting of —CH₂—CH₂—, —CH₂—, —CH₂—CH₂—CH₂—, and —CH₂—CH₂—CH₂—CH₂—, optionally substituted once, twice, or three times by halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl; wherein
R^{I} is alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{II} and R^{III} are independently from each other selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or
R^{II} and R^{III} together form a five-membered or six-membered heterocycloalkyl, heterocycloalkenyl or heteroaryl group, wherein each group is optionally substituted once, twice, or three times;
R^{IV} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{V} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R^{VI} is hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times;
R₃₁, R₃₂, R₃₃, and R₃₄ are each independently selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₃₅ is alkyl, hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above; 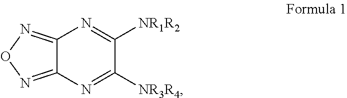
wherein
R₁ and R₂ are each independently selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or
R₁ and R₂ together form a five-membered or six-membered heterocycloalkyl, heterocycloalkenyl or heteroaryl group, which is optionally substituted once, twice, or three times;
R₃ and R₄ are each independently selected from the group consisting of hydrogen, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; or
R₃ and R₄ together form a five-membered or six-membered heterocycloalkyl, heterocycloalkenyl or heteroaryl group, which is optionally substituted once, twice, or three times; 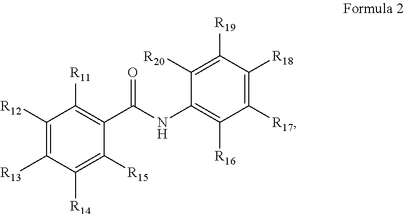
wherein
R₁₁, R₁₂, R₁₃, R₁₄, R₁₅, R₁₆, R₁₇, R₁₈, R₁₉ and R₂₀ are each independently selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above; 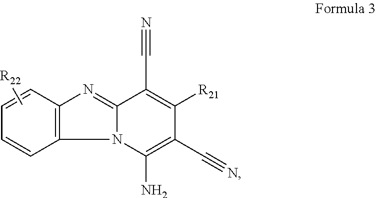
wherein
R₂₁ is selected from the group consisting of hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₂₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above; 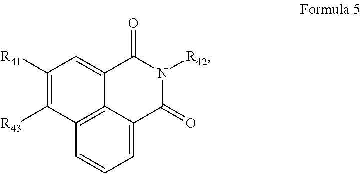
wherein
R₄₁ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;
R₄₂ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above;
R₄₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above; 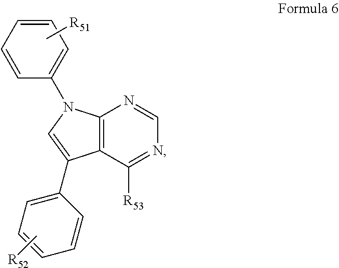
wherein
R₅₁ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₅₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₅₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{III}, R^{IV}, R^{V} and R^{VI} are defined as above; 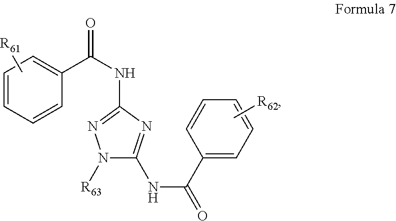
wherein
R₆₁ represents 0, 1, or 2 substituents that are independently selected from the group consisting of halogen, —OH, —OR^{I}, —SH,_NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₆₂ represents 0, 1, or 2 substituents that are independently selected from the group consisting of halogen, —OH, —OR^{I}, —SH, —NR^{II}R^{III}, —NO², —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₆₃ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above; 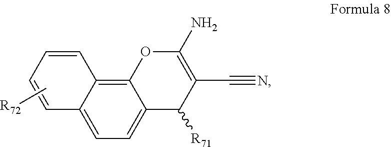
wherein
R₇₁ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}_{, R}^{IV}, R^{V} and R^{VI} are defined as above;
R₇₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above; 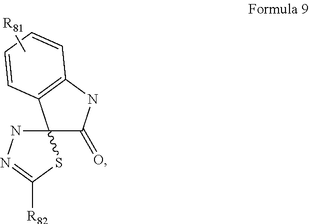
wherein
R₈₁ represents 0, 1, or 2 substituents that are independently selected from the group consisting of halogen, —OH, —OR^{I}, —SH, ^{—NR}^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
R₈₂ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} defined as above; 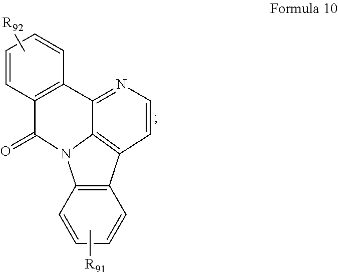
wherein
R₉₁ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above; and
R₉₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein each group is optionally substituted once, twice, or three times; wherein R^{I}, R^{II}, R^{III}, R^{IV}, R^{V} and R^{VI} are defined as above;
and
pharmaceutically acceptable salts of any one of formulae 1 to 10.

2. The compound according to claim 1, wherein the compound is selected from the group consisting of: 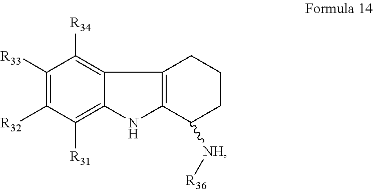
wherein
R₃₁, R₃₂, R₃₃, and R₃₄ are defined as in claim 1;
R₃₆ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above; 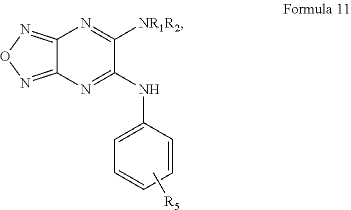
wherein
R₁ and R₂ are defined as in claim 1;
R₅ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as in claim 1; 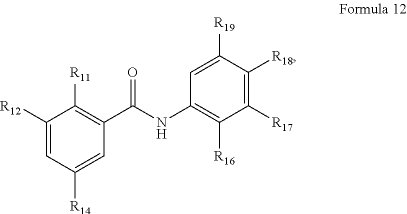
wherein R₁₁, R₁₂, R₁₄, R₁₆, R₁₇, R₁₈, and R₁₉ are defined as in claim 1; 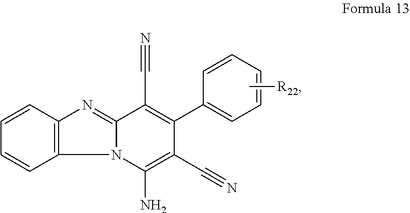
wherein R₂₂ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, hetero cyclo alkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as in claim 1; 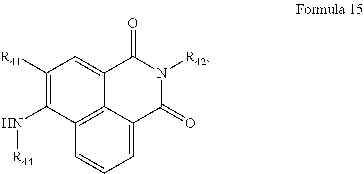
wherein
R₄₁ and R₄₂ are defined as in claim 1;
R₄₄ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}R^{V} and R^{VI} are defined as above; 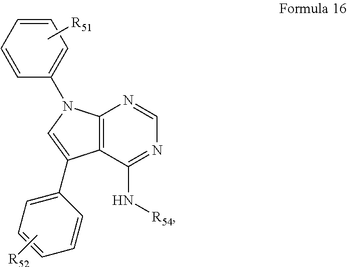
wherein
R₅₁ and R₅₂ are defined as in claim 1;
R₅₄ is hydrogen, halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, or heteroaralkynyl, wherein each group is optionally substituted once, twice, or three times; wherein R^{I}, R^{II}, R^{III}, R^{IV}, R^{V} and R^{VI} are defined as above; 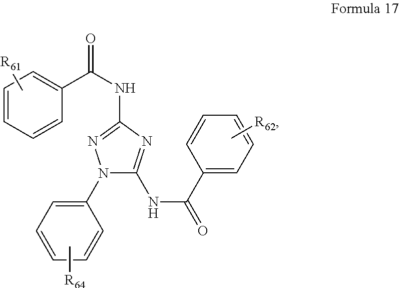
wherein
R₆₁ and R₆₂ are defined as in claim 1;
R₆₄ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as in claim 1; 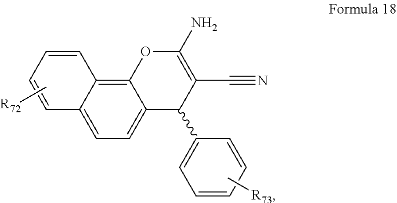
R₇₂ is defined as in claim 1;
R₇₃ represents 0, 1, or 2 substituents that are independently selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as in claim 1; 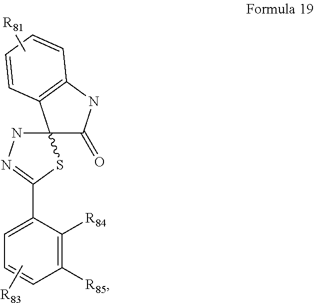
wherein
R₈₁ is defined as in claim 1;
R₈₃ represents 0, 1, or 2 substituents that are independently selected from the group consisting of halogen, —OH, —OR^{I}, —NR^{II}R^{III}, —NO², —SH, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein R^{I}, R^{II}, R^{III}, R^{IV}, R^{V} and R^{VI} are defined as in claim 1;
R₈₄ and R₈₅ are each independently from the other selected from the group consisting of halogen, —OH, —OR^{I}, —SH, —NR^{II}R^{III}, —NO₂, —SO₂, —CO—OR^{IV}, —CO—NHR^{V}, —SO₂—NHR^{VI}, alkyl, heteroalkyl, haloalkyl, cycloalkyl, heterocycloalkyl, alkenyl, heteroalkenyl, cycloalkenyl, heterocycloalkenyl, alkynyl, heteroalkynyl, cycloalkynyl, heterocycloalkynyl, an alicyclic system, aryl, aralkyl, aralkenyl, aralkynyl, heteroaryl, heteroaralkyl, heteroaralkenyl, and heteroaralkynyl, wherein, R^{I}, R^{II}, R^{III}, R^{IV}, R^{V} and R^{VI} are defined as in claim 1; or
R₈₄ and R₈₅ together form a five-membered or six-membered alkenyl, aryl, heterocycloalkenyl or heteroaryl group, which is condensed with the phenyl group to which R₈₄ and R₈₅ are attached and which is optionally substituted once, twice, or three times; 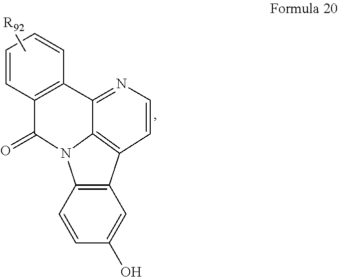
wherein R₉₂ is defined as in claim 1;
and
pharmaceutically acceptable salts of any one of formulae 11 to 20.

3. The compound according to claim 1, wherein the compound is selected from the group consisting of: 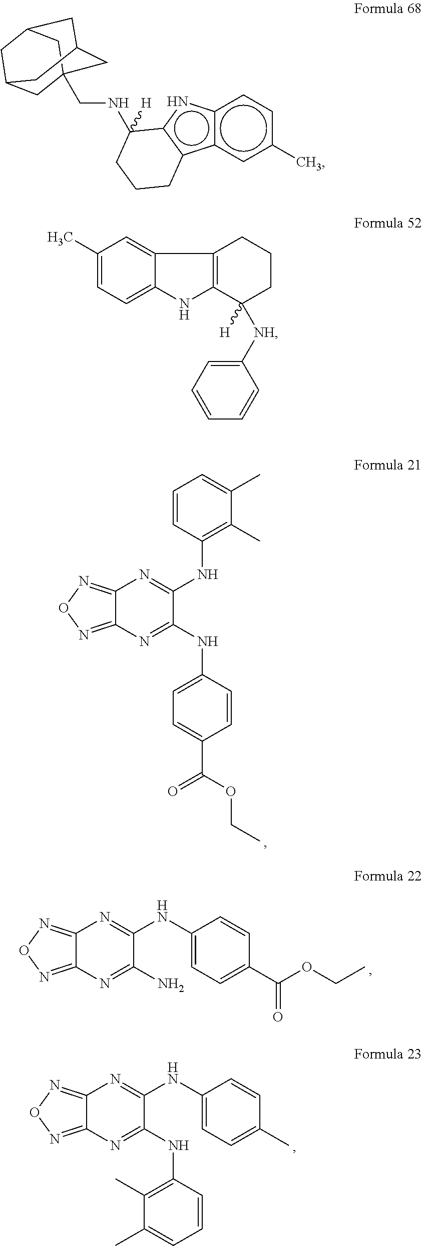 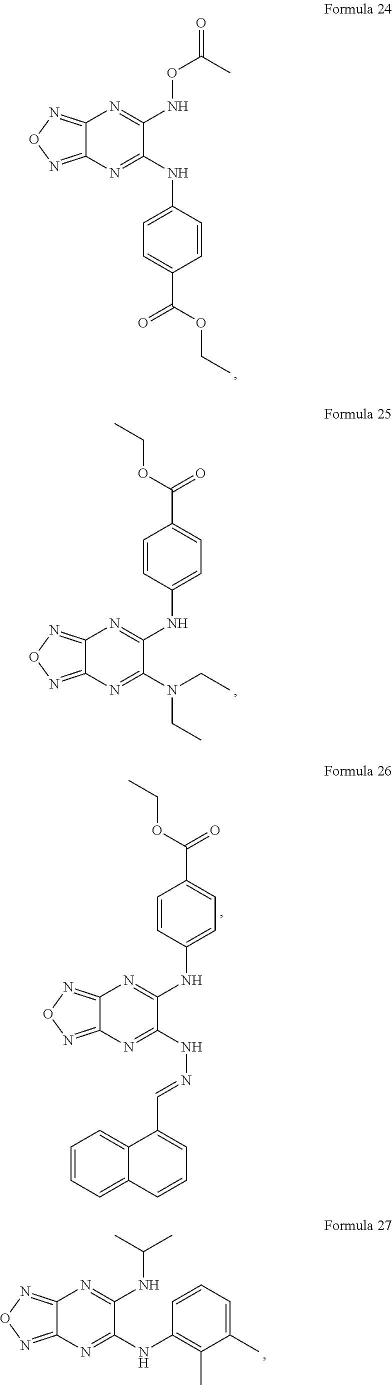 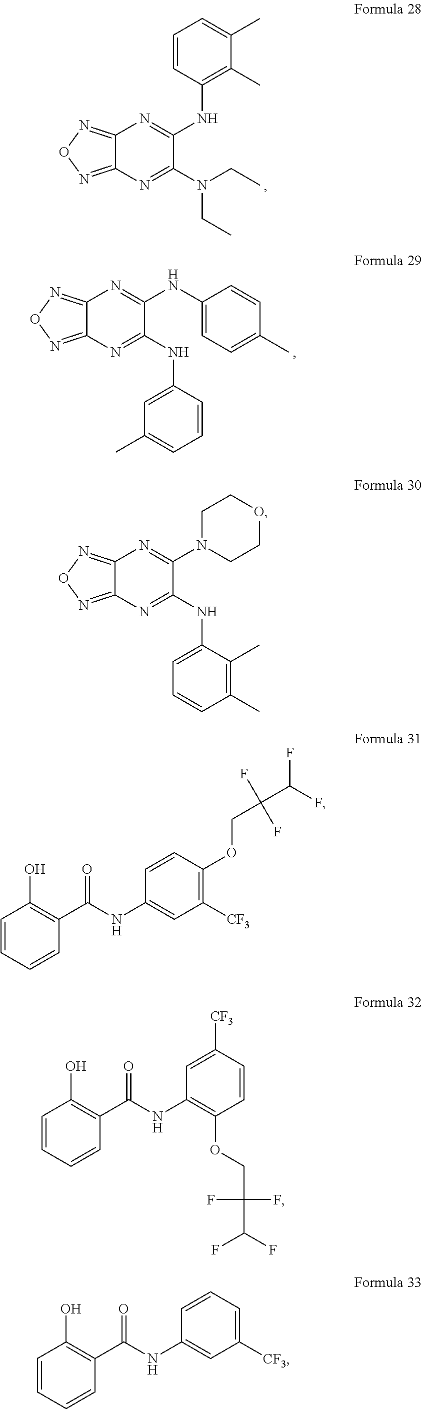 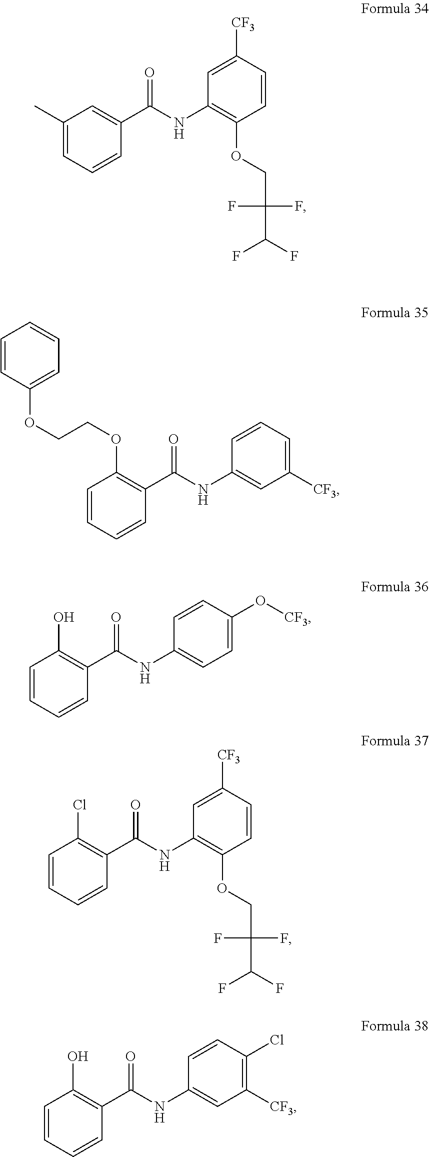 [SMILES: Cc1cccc(C(=O)Nc2cc(C(F)(F)F)ccc2OCC(F)(F)C(F)F)c1 ; O=C(Nc1cccc(C(F)(F)F)c1)c1ccccc1OCCOc1ccccc1 ; O=C(Nc1ccc(OC(F)(F)F)cc1)c1ccccc1O ; O=C(Nc1cc(C(F)(F)F)ccc1OCC(F)(F)C(F)F)c1ccccc1Cl ; O=C(Nc1ccc(Cl)c(C(F)(F)F)c1)c1ccccc1O] 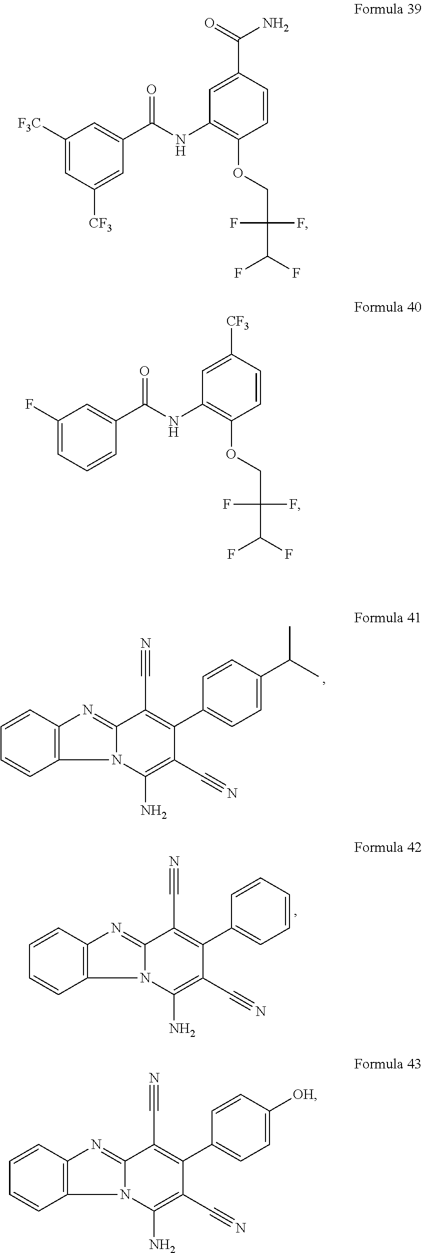 [SMILES: NC(=O)c1ccc(OCC(F)(F)C(F)F)c(NC(=O)c2cc(C(F)(F)F)cc(C(F)(F)F)c2)c1 ; O=C(Nc1cc(C(F)(F)F)ccc1OCC(F)(F)C(F)F)c1cccc(F)c1 ; CC(C)c1ccc(-c2c(C#N)c(N)n3c(nc4ccccc43)c2C#N)cc1 ; N#Cc1c(-c2ccccc2)c(C#N)c2nc3ccccc3n2c1N ; N#Cc1c(-c2ccc(O)cc2)c(C#N)c2nc3ccccc3n2c1N] 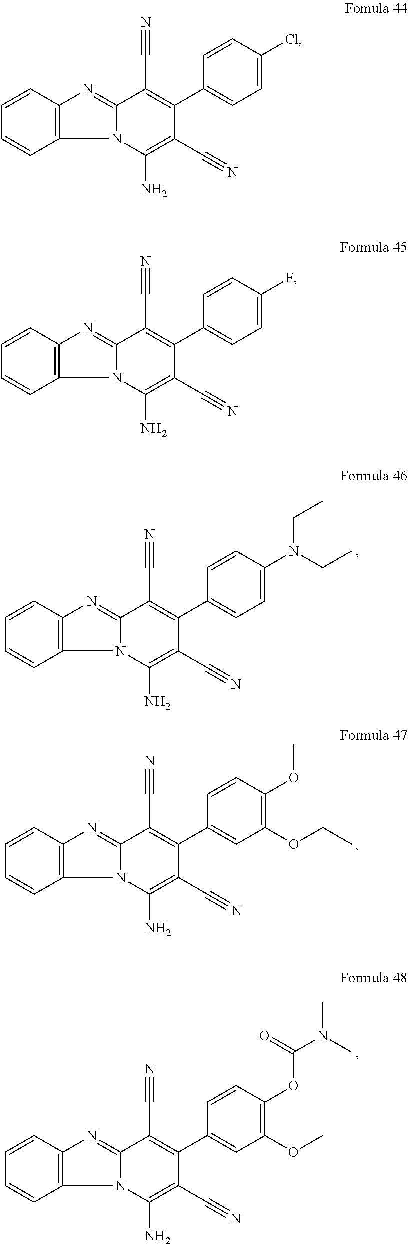 [SMILES: N#Cc1c(-c2ccc(Cl)cc2)c(C#N)c2nc3ccccc3n2c1N ; N#Cc1c(-c2ccc(F)cc2)c(C#N)c2nc3ccccc3n2c1N ; CCN(CC)c1ccc(-c2c(C#N)c(N)n3c(nc4ccccc43)c2C#N)cc1 ; CCOc1cc(-c2c(C#N)c(N)n3c(nc4ccccc43)c2C#N)ccc1OC ; COc1cc(-c2c(C#N)c(N)n3c(nc4ccccc43)c2C#N)ccc1OC(=O)N(C)C] 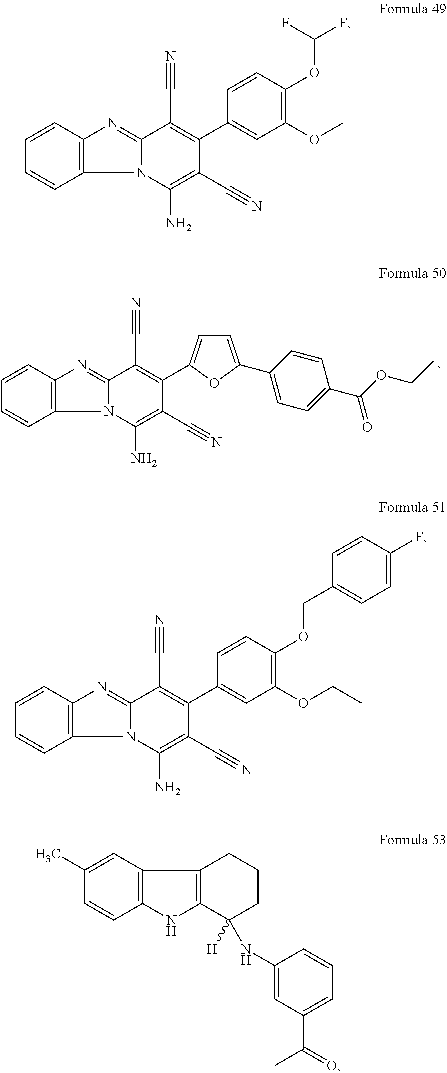 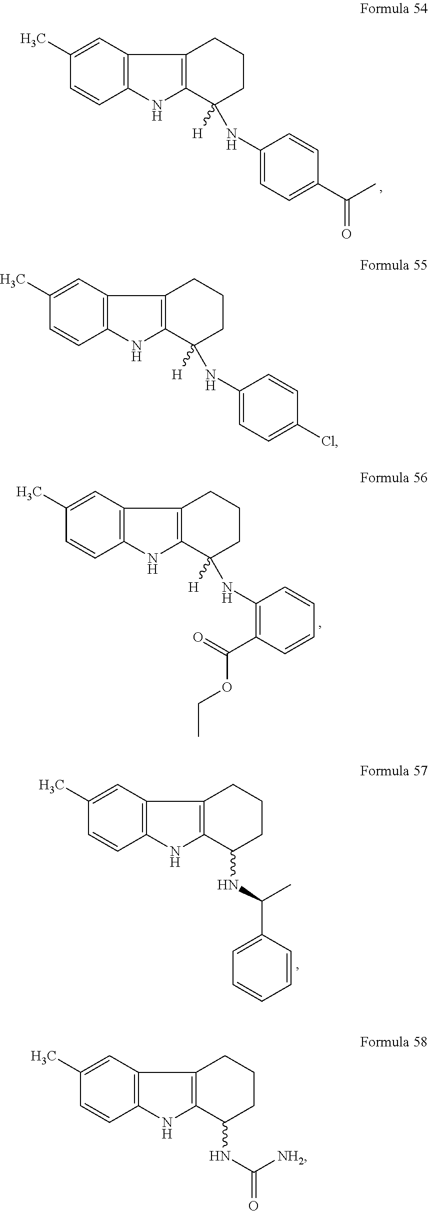 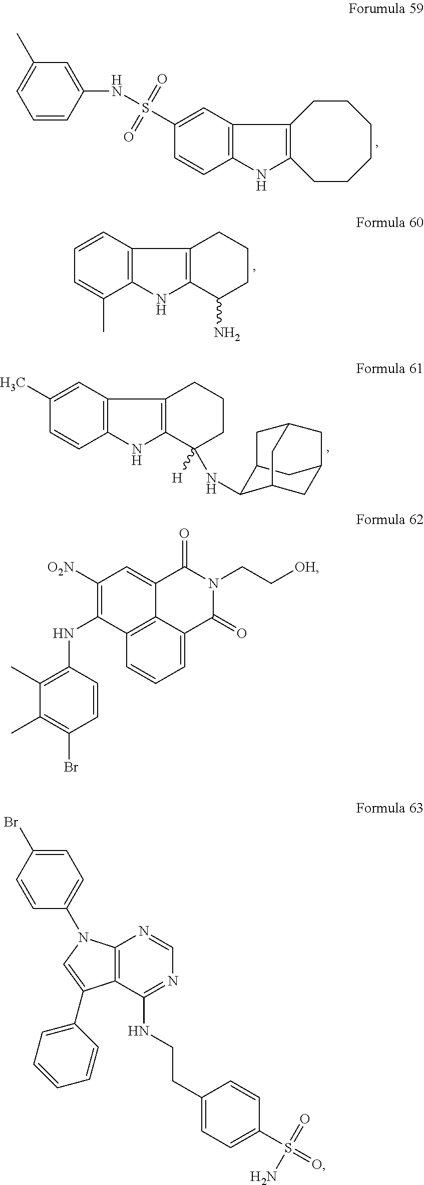 [SMILES: Cc1cccc(NS(=O)(=O)c2ccc3[nH]c4c(c3c2)CCCCCC4)c1 ; Cc1cccc2c3c([nH]c12)C(N)CCC3 ; Cc1ccc2[nH]c3c(c2c1)CCCC3NC1C2CC3CC(C2)CC1C3 ; Cc1c(Br)ccc(Nc2c([N+](=O)[O-])cc3c4c(cccc24)C(=O)N(CCO)C3=O)c1C ; NS(=O)(=O)c1ccc(CCNc2ncnc3c2c(-c2ccccc2)cn3-c2ccc(Br)cc2)cc1] 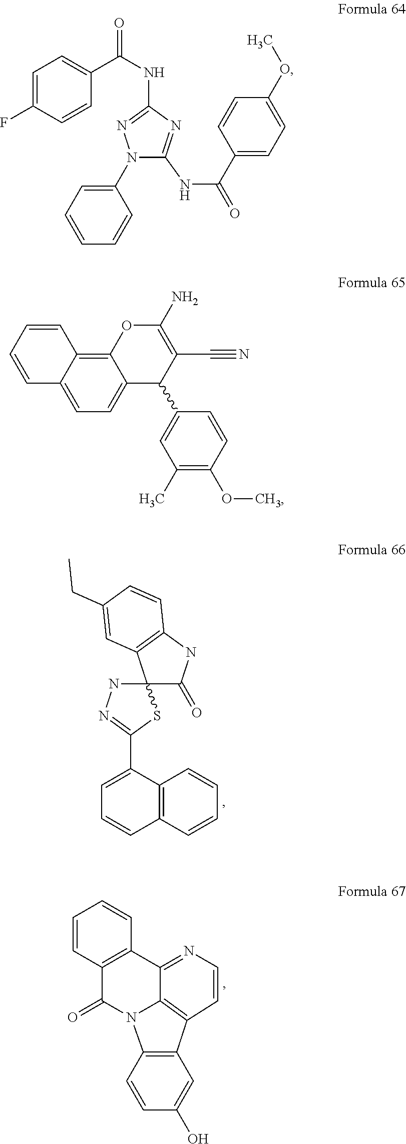 [SMILES: COc1ccc(C(=O)Nc2nc(NC(=O)c3ccc(F)cc3)nn2-c2ccccc2)cc1 ; COc1ccc(C2C(C#N)=C(N)Oc3c2ccc2ccccc32)cc1C ; CCc1ccc2c(c1)C1(NN=C(c3cccc4ccccc34)S1)C(=O)N2 ; O=c1c2ccccc2c2nccc3c4cc(O)ccc4n1c32] 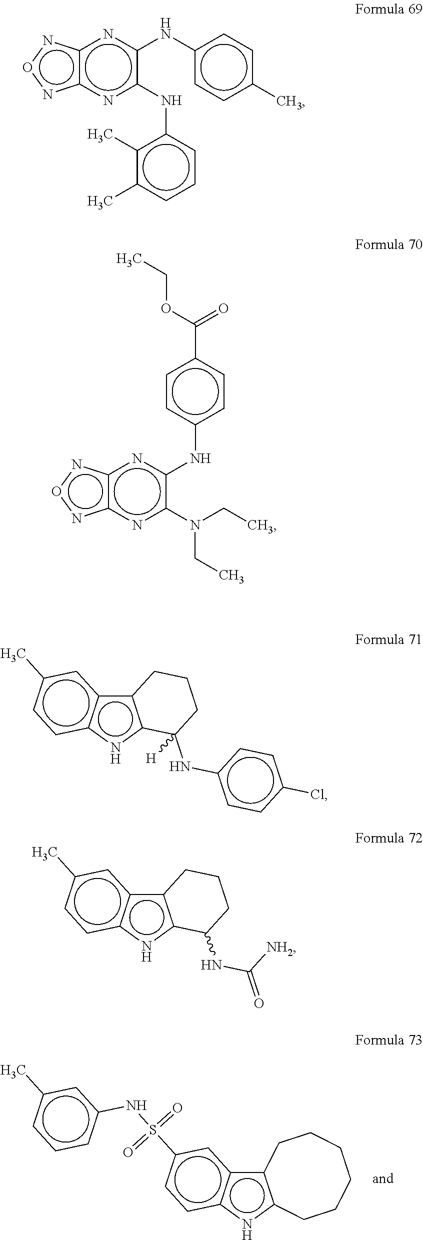 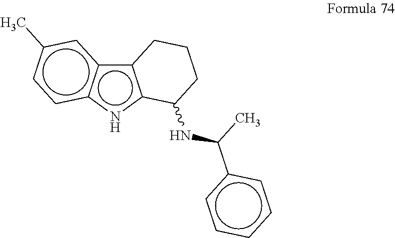
and pharmaceutically acceptable salts of any one of formulae 21 to 74.

4. A compound according to claim 1 for use in medicine.

5. A compound according to claim 1 for use in the treatment of cancer or for use in the induction of cell regeneration.

6. The compound according to claim 5, wherein said cancer is selected from the group consisting of breast cancer, kidney carcinoma, prostate cancer, cervical cancer, ovarian cancer, hepatocellular and squamous cell carcinoma, soft tissue sarcoma, leukemia, glioma, and multiple myeloma.

7. The compound according to claim 5, wherein said compound is for use in the induction of cell regeneration in aging tissue, in tissue damaged by a chronic disease, or in tissue damaged by acute injury.

8. The compound according to claim 7, wherein said chronic disease is selected from the group consisting of liver cirrhosis, damages caused by chemotherapy, chronic HIV virus infection, bone marrow failure syndromes, chronic wounds, and ulcerative colitis.

9. The compound according to claim 7, wherein said acute injury is selected from the group consisting of burns, lacerations, cuts, invasive surgical interventions, injuries caused by chemo- or radiotherapy, and any other type of tissue damage.

10. The compound according to claim 7, wherein said tissue is selected from the group consisting of liver, skin, cartilage, bone marrow, intestine, and muscle.

11. A pharmaceutical composition comprising a compound according to claim 1.

12. An article of manufacture comprising:
(a) a packaging material;
(b) a compound according to claim 1; and
(c) a label or packaging insert contained within the packaging material indicating that patients receiving treatment with said compound can be treated for cancer and/or indicating that cell regeneration is induced in patients receiving treatment with said compound.
